(19) United States
(12) Patent Application Publication
(10) Pub. No.: US 2019/0021608 A1
(43) Pub. Date: Jan. 24, 2019

(54) CD206+ Macrophage-Specific Molecular Imaging Probe Compositions and Methods and the Noninvasive Quantification of Arterial Wall Macrophage Infiltration in Humans

(71) Applicant: Navidea Biopharmaceuticals, Inc., Dublin, OH (US)

(72) Inventors: Frederick O. Cope, Dublin, OH (US); David Allen Ralph, Columbus, OH (US); Bonnie Chandler Abbruzzese, Dublin, OH (US)

(21) Appl. No.: 15/984,282

(22) Filed: May 18, 2018

Related U.S. Application Data
(60) Provisional application No. 62/509,010, filed on May 19, 2017.

(51) Int. Cl. A61B 5/02; A61K 51/02

(52) U.S. Cl. CPC A61B 5/02007; A61K 51/025

(57) ABSTRACT

Described herein are compositions and methods for diagnosing, imaging, and quantifying non-calcified atherosclerotic plaque. Embodiments of compositions described herein comprise mannosylated dextran compounds along with other carbohydrate molecules comprising, for example, a diagnostic agent and a diagnostic moiety. The compounds and methods embodied herein produce superior localization and results for imaging, anatomically locating, and quantifying non-calcified atherosclerotic plaque.

### CROSS-REFERENCE TO RELATED APPLICATIONS

This application claims the benefit of U.S. Provisional Application No. 62/509,010 filed on May 19, 2017. The entire contents of this application is incorporated herein by reference in its entirety.

### DETAILED DESCRIPTION

Herein is disclosed is the use of mannosylated dextran molecular constructs as exemplified by ^{99m}Tc-tilmanocept, a class of synthetic high affinity ligands for the macrophage mannose receptor (CD206), as imaging agents with utility for visualization and quantitation of macrophage mediated inflammation in non-calcified atherosclerotic plaques. Inflammatory processes in non-calcified atherosclerotic plaques is the underlying pathobiology leading to myocardial infarctions, most ischemic strokes and a number of other vascular conditions that together are the leading cause of death and disability worldwide.

Atherosclerosis is a chronic, progressive, systemic, maladaptive, inflammatory syndrome in which activated macrophages contribute significantly to the initiation, maintenance, growth and eventual rupture of atherosclerotic lesions: plaques in the walls of arteries ^{[1]}. Rupture (or erosion) of atherosclerotic plaques causes blood clots that block blood flow (emboli leading to infarctions) resulting in the large majorities of ischemic strokes (IS) ^{[2]} and heart attacks (myocardial infarctions, MIs) ^{[3]}. As such, atherosclerosis is the underlying leading cause of death and disability in the USA and worldwide ^{[4, 5]}. Alternative atherosclerosis imaging modalities currently in common practice are either highly invasive or measure physical attributes of some atherosclerotic plaques, such as calcium content or stenosis, that are clearly not linked to plaque inflammation and only indirectly linked to plaque rupture risk ^{[6]}. Direct imaging of the inflammatory microenvironments of atherosclerotic plaques as enabled by the current invention provides key information not accessible by other imaging modalities, enabling more accurate assessment of atherosclerotic plaques relative to the extent of disease, risks of plaque rupture, and likely for monitoring the effectiveness of atherosclerosis directed therapies.

Atherosclerosis can cause clinical symptoms by two mechanisms: Reducing blood flow by narrowing the luminal diameter of arteries (stenosis), or by blocking blood flow through blood clots arising from disrupted atherosclerotic plaques leading to emboli and resulting infarctions (local tissue death). Of these two mechanisms, blood clot emboli and their resulting infarctions cause, by far, the greatest portion of death and disability that is attributable to atherosclerosis. According to the Centers for Disease Control (CDC, 2017), every year 735,000 Americans suffer MIs from which about half (370,000) will die. Similarly, approximately 690,000 Americans suffer an IS each year, a large proportion of which were caused by emboli resulting from the rupture of atherosclerotic plaques, frequently residing in carotid arteries. More than 100,000 Americans die each year from these strokes. Atherosclerosis, which is a systemic condition, can also cause significant pathology elsewhere in the body. Atherosclerosis causes peripheral artery disease (PAD) ^{[7]}, which is a significant cause of disability in the elderly, impacting nearly 12% of the Medicare population ^{[8]}. Atherosclerosis can cause vascular dementia and can contribute to kidney disease ^{[9]}. All told, the consequences of atherosclerosis negatively impact millions of Americans and are the leading cause of death in the US. According to the World Health Organization, each year worldwide 8.75 million people die of heart disease, the very large majority of which was caused by atherosclerosis. In addition, 6.24 million persons worldwide die from IS. Compositions described herein can also be used as a method of testing for inflammation of the type observed in atherosclerosis. Using the compositions as described herein can allow for the monitoring of the correct level of anti-inflammatory substance to be given to a subject. This can avoid over-administration of anti-inflammatory substances and allows for variation and correction of the amount and/or titrate the amount of substance administered to alleviate the inflammation.

Certain populations of patients suffer disproportionally from the adverse consequences of atherosclerosis. One such population of patients consists of persons living with infections of the Human Immunodeficiency Virus (HIV+ persons). Beginning in the mid-1990's, the development and availability of Highly Active Anti-Retroviral Therapy (HAART) greatly reduced the mortality rate of individuals infected with Human Immunodeficiency Virus (HIV) ^{[10]} The advent of HAART increased the life expectancy of patients with HIV related Acquired Immunodeficiency Syndrome (AIDS) from a few months or years to decades. However, HAART therapy does not eradicate (cure) HIV from the patient's body. Instead, HIV persists in HAART resistant reservoir where it is controlled and contained by HAART but not eliminated ^{[11]}. This persistent HIV infection leads to chronic inflammation that manifests in many forms, including increased cardiovascular inflammation ^{[12, 13]}. This increased inflammation causes accelerated development of atherosclerosis in HIV infected persons on HAART ^{[14-17]}, which in turn leads to increased risks from IS ^{[18, 19]} and MIs ^{[20-22]} in this patient population. HIV patients are thus an excellent model for investigation of the etiology of the atherosclerosis condition in the general population as well as in HIV and other subjects.

Macrophages are highly adaptive. Responding to stimuli from their surroundings, they can adopt a wide variety of activated phenotypic states ^{[23, 24]}. When originally described, activated macrophage phenotypes were divided into two classes: classically activated macrophages (referred to as M1), which were viewed as highly pro-inflammatory, and alternatively activated macrophages (referred to as M2), which were viewed as being immunosuppressive and involved in wound healing. The expression level of the genes for CD206 can vary dramatically in macrophages due to their activated phenotypic state is CD206 ^{[25]} (from Q-Path). Although a soluble form of CD206 is known ^{[26, 27]}, CD206 most typically occurs as an approximately 175 kDₐ ^{[28]}, transmembrane ^{[29]}, glycosylated ^{[30]}, C-type lectin ^{[31]} with eight extracellular sugar binding domains that mediate high affinity, multivalent binding to ligands displaying multiple mannose moieties. Once a high mannose ligand has bound CD206, it is internalized by receptor mediated endocytosis. After the CD206/ligand complex has been internalized, CD206 releases its ligand and is recycled to the cell surface ^{[32, 33]}. Thus, CD206 ligands can be “loaded” into a cell with each CD206 receptor capable of internalizing multiple ligand molecules. Originally, high levels of CD206 expression were thought to be restricted to activated macrophages that had adopted an M2 (immunosuppressive) phenotype. However, it was determined that a dichotomous differentiation of activated macrophage phenotypes as being either M1 or M2 is overly simplistic and does not represent the true plasticity of macrophage responses to environmental stimuli. Because of the critical roles that macrophages play in the development, progression and ultimate resolution of atherosclerotic plaques, the phenotypic states of activated macrophages in atherosclerotic plaques has been extensively investigated^{[34-37]}. Many different activated phenotypes have been described for macrophages residing within atherosclerotic plaques with macrophages exhibiting different activated phenotypes being observed within a single atherosclerotic plaque simultaneously. But, prior to the invention, the prior art believed that CD206 expression was limited in atherosclerotic plaques and that any amount of expression was insufficient to allow for diagnostic imaging or visualization as set forth herein. The inventors unexpectedly determined that was not the case as discussed herein. Prior to the current invention, the prior art did not fully consider the levels of CD206 expression in the context of the instant invention. It was previously believed that CD206 expression was confined to M2-like (immunosuppressive) activated phenotypes. A more recent study by Cochain et al^{[38]} that was conducted after the studies described in the examples were completed, directly evaluated CD206 expression in atherosclerotic plaques in Ldlr^{-/-} mice, a murine model of atherosclerosis that closely mimics human atherosclerosis. This study used deep mRNA sequencing of individual cells isolated from atherosclerotic plaques to evaluate the activated phenotypes with plaque macrophages. It was observed that macrophage phenotypes fell broadly into one of three classes: An M1-like class, an M2-like class and a third previously undescribed class representing about 20% of total macrophages. Unexpectedly, nearly all of the M2-like and a majority of the M1-like macrophages highly expressed CD206. Thus, contrary to expectations based on previous observations of elevated CD206 expression confined to M2-like cells, the majority of macrophages in the examined atherosclerotic plaques, independently of their pro-inflammatory or immunosuppressive phenotype, highly expressed CD206.

While many types of immune cells participate in the development and progression of atherosclerotic plaques, the involvement of macrophages is highly conspicuous with macrophages being key drivers of the chronic inflammation that is characteristic of this disease ^{[39, 40]}. Following an injury to the arterial endothelium, low density lipoproteins (LDL) invade the endothelium and become oxidized, initiating an inflammatory response that attracts monocytes. These monocytes ingest the oxidized LDL and become macrophage “foam cells” that further propagate the inflammatory response by secreting proinflammatory cytokines ^{[38, 41]}. Eventually, the foam cells die or undergo apoptosis creating the lipid rich necrotic core ^{[42]}. The necrotic core further attracts macrophages that become activated in response to the local inflammatory microenvironment. The end result of this progression is a plaque that is vulnerable to rupture (or erosion) ^{[43, 44]}. Rupture vulnerable plaques are characterized by large lipid cores covered by a thin fibrous cap ^{[45-51]}. Such plaques have been termed thin-cap fibroatheromas. Activated macrophages are highly numerous in this category of plaque. There are two important anatomical features of plaques that are vulnerable to rupture: they have limited (spotty) deposits of calcium (microcalcifications) and, because of arterial wall remodeling, they are frequently not associated with clinically significant stenosis ^{[52, 53]}.

Not all atherosclerotic plaques eventually rupture, creating infarction causing emboli. Instead, some plaques can resolve. Plaques can undergo a macrophage-mediated macrocalcification that stabilizes the plaques from rupture ^{[34, 36, 54, 55]}. Macrocalcification is associated with reduced inflammation and plaque stability. Highly calcified plaques are unlikely to rupture and have low densities of activated macrophages ^{[56-59]}. The inventors determined that a macrophage directed imaging agent will preferentially identify non-calcified plaques with active inflammation, the class of atherosclerotic plaques that are most vulnerable to rupture.

Prior to the invention and in current practice, several noninvasive imaging modalities have been used to assess atherosclerosis. The most significant deficiency of these alternative imaging modalities for atherosclerosis is that they do not directly assess inflammatory processes within atherosclerotic plaques ^{[58, 60-65]}. X-ray based computed tomography (CT) is the most commonly used imaging modality to evaluate patients for atherosclerotic risks. CT images detect x-ray opacity, which readily enables the detection of calcium deposits, which are then reported as a calcium score. The higher the calcium score, the greater the amount of calcium. Thus, CT mostly detects stable plaques that are unlikely to rupture. The presence of calcified plaques is used to infer the presence elsewhere of non-calcified plaque that could potentially rupture. In truth, persons with low calcium scores rarely die from CVD events ^{[66]}, probably because they have low non-calcified plaques burdens too. In addition, calcium score is strongly correlated with risk of future CVD events. However, when added to a multivariate risk model that included a questionnaire and commonly and easily accessible clinical findings, calcium score only modestly improved the discriminatory accuracy of the combined model (c-statistic: 0.744 vs. 0.755) ^{[67]}. In a different but similar study, the difference between the c-statistics before and after addition of calcium score was greater, but the c-statistic of the combined model was still only 0.755 ^{[68]}. While these results were highly significant, adding a calcium score to an established risk model resulted in reclassification of relatively few people into low-risk (don't treat) or high-risk (do treat) groups. In another study ^{[69]}, a combined risk model similar to the one used in the other studies placed 31% of participants in the high-risk group while partitioning 24% of participants who went on to experience a CVD event into the low-risk group. Clearly, there continues to be a need for more accurate risk stratification.

CT can also be combined with a contrast agent to perform CT angiography (CTA). CTA detects arterial stenosis, and to a limited extent, non-calcified plaque but not inflammation. It is most commonly used to evaluate coronary arteries for evidence of coronary artery disease (CAD). Like CT, CTA has high negative predictive value for cardiovascular disease (CAD). However, CTA has relatively low positive predictive value (PPV) and specificity for detecting CAD. In one study, the PPV and specificity of CTA for CAD were 68% and 50% respectively ^{[70]}. In another study, the PPV on a per-patient basis was only 40% ^{[71]}. As described herein, using ^{99m}Tc-tilmanocept based imaging, through the direct observation of atherosclerotic plaque inflammation, is not only unexpectedly superior to these techniques, but offers additional unexpected benefits.

Magnetic resonance imaging (MRI) is a rapidly evolving technology and imaging modality ^{[72]}. It can measure arterial stenosis, and interestingly, MRI has the potential to detect intra-plaque hemorrhage, which is a feature of many late stage vulnerable plaques. MRI is used to evaluate atherosclerotic plaques of the carotid arteries ^{[73]}. However, technical issues are preventing MRI evaluation of atherosclerosis at other sites from becoming a standard of care at this time. More importantly, MRI can detect only a portion of non-calcified plaques. MRI does not image inflammation directly. As described herein, ^{99m}Tc-tilmanocept (along with the other embodiments of compositions described herein) imaging unexpectedly has a much greater sensitivity for inflamed plaques, including those detected by MRI.

Ultrasound (ULS) imaging of the carotid arteries is in common practice to evaluate atherosclerosis of the carotid arteries ^{[74]}. ULS measures calcium content, blood flow and stenosis. Furthermore, carotid ULS can assist physicians and other users that may administer the compositions described herein in identifying subjects at risk for carotid plaque rupture and ischemic stroke, and because atherosclerosis is a systemic condition, for CVD risks generally ^{[75]}. ULS images carotid plaque morphology but has limited ability to evaluate key anatomical features of vulnerable plaque. Directly detecting and measuring plaque inflammation, ^{99m}Tc-tilmanocept imaging provides key information about carotid atherosclerotic plaques relative to both extent and accompanying risk than is not accessible by ULS.

Finally, there are two diagnostic radiopharmaceutical imaging agents, both labeled with ¹⁸fluoride ([18F]), that have been used to image atherosclerotic plaques in investigational studies. Both are used to enable positron emission tomography (PET) or in combination with x-ray based CT, PET/CT imaging. These imaging agents are sodium fluoride ([18F]Na) and fluorodeoxyglucose ([18F]FDG). [18F]Na localizes to calcium deposits such as occur in bone and in calcified deposits in atherosclerotic plaques. In one of the examples described below, [18F]Na based PET imaging was used to discriminate between calcified and non-calcified atherosclerotic plaques.

[18F]FDG identifies tissues with higher levels of glucose metabolism such as occur in many tumors. Other anatomical sites with elevated glucose metabolism include areas of active inflammation. In a study of 8 individuals whose carotid arteries were imaged by [18F]FDG-PET, [18F]FDG localized to symptomatic (inflamed) plaques that were further shown to be densely populated with CD68 expressing macrophages [85]. Arterial uptake of [18F]FDG is correlated with future CVD events [86]. In addition, after 12 weeks of high-dose statin therapy (but not low dose therapy), FDG-PET imaging of the aorta and coronary arteries revealed a 14% decrease in inflammation (localization of [18F]FDG relative to background) [87]. Although not in common current medical practice use, [18F]FDG-PET imaging shares a significant attribute with the imaging agent described in the current invention in that both imaging agents localize to inflamed, non-calcified atherosclerotic plaques that include those plaques that are most vulnerable to rupture. However, there are three reasons why the imaging agent disclosed in the current invention will be preferred to [18F]FDG for imaging inflamed, non-calcified atherosclerotic plaques. First, [18F]FDG localization is not specific to areas involved in inflammation. [18F]FDG-PET imaging detects areas of elevated glucose metabolism. With few exceptions, all tissues metabolize glucose, which creates an imaging background with an associated signal to noise issue. Second, some organs have naturally high levels of glucose metabolism in healthy individuals. Examples of organs with naturally high levels of glucose metabolism include the brain and the heart. High levels of natural glucose metabolism in the heart creates a significant barrier to the utilization of [18F]FDG to image inflamed, non-calcified plaques in coronary arteries the rupture of which cause MIs. To lessen the severity of this barrier, patients scheduled for [18F]FDG cardiac imaging must be placed on a special diet for some period prior to undergoing the imaging procedure. Finally, [18F] is manufactured with a cyclotron and has a half-life of 110 minutes. This creates a logistical barrier for the availability of [18F]FDG (and [18F]Na), largely limiting its access to institutions with their own cyclotrons.

An example of an imaging agent embodied by the current invention is ^{99m}technetium (^{99m}Tc) labeled tilmanocept. Tilmanocept is a member of a class of molecular constructs (compounds) that are intentionally designed as high affinity ligands for CD206 ^{[76, 77]}. Tilmanocept's dissociation constant for CD206 is 3×10⁻¹¹. It does not significantly localize to tissues of the heart or arteries in the absence of inflammatory processes such as occur in non-calcified atherosclerotic plaques, whereas it was determined that the compositions of the instant invention demonstrated unexpectedly superior localization in atherosclerotic non-calcified plaque. Therefore, within the context of imaging atherosclerosis, ^{99m}Tc-tilmanocept enabled imaging is specific for aggregations of CD206 expressing macrophages such as occurs in non-calcified plaques and is not burdened by nonspecific localization to non-involved tissues such as is observed with [18F]FDG-PET imaging. In some embodiments, ^{99m}Tc-Tilmanocept localizations (or localizations of other compositions as disclosed herein) can be visualized using gamma camera planar imaging or using single-photon emission computed tomography (SPECT) or SPECT/CT imaging. Since ^{99m}Tc has a half-life of 6 hours, it can be produced at a central location and distributed regionally, permitting institutions without the capability to make ^{99m}Tc to have access to this isotope and ^{99m}Tc-tilmanocept.

While practitioners are likely to prefer ^{99m}Tc-tilmanocept to [18F]FDG for imaging non-calcified atherosclerotic plaques, the experience with [18F]FDG indicated that [18F]FDG-PET imaging can: 1) detect and quantify the location, extent and plaque volume of non-calcified and inflamed atherosclerotic plaques, 2) predict future cardiovascular disease event such as MI and IS, and 3) monitor the effectiveness of atherosclerosis directed therapies by quantifying changes in non-calcified plaque volume over the course of treatment. ^{99m}Tc-Tilmanocept based imaging unexpectedly has the same attributes and performance characterizations without the non-inflammation associated localization seen with [18F]FDG. Because [18F]FDG lacks the localization of the instant invention and is a higher energy emitter, there are radiation and health issues associated therewith.

^{99m}Tc-tilmanocept is described in Application No. PCT/US2015/041036, which is hereby incorporated by reference in its entirety. The synthesis of tilmanocept has been previously described ^{[78, 79]}. Briefly, beginning with a 10 kDa dextran backbone, amine terminated molecular leashes are added to the dextran's glucose moieties. To these leashes, approximately 5 moieties of the chelating agent, DTPA, and approximately 17 mannose moieties are attached to create the approved commercial product, Lymphoseek® (FIG. 1). Lymphoseek is an FDA approved diagnostic imaging agent for indications that are unrelated to atherosclerosis and CVD (i.e. identification of sentinel lymph nodes during surgeries to remove solid tumor cancers). After synthesis, residual leashes remain on tilmanocept that are available for further chemical additions. For some experiments described in the examples, a fluorescent derivative of tilmanocept was synthesized through addition of 1-2 Alexa-488 moieties to unoccupied leashes as previously described ^{[80, 81]}.

Abbreviations: ^{99m}Tc, metastable technetium with an atomic weight of 99; SPECT, single photon emission computed tomography; CT, computed tomography; HIV, Human Immunodeficiency Virus; HIV+, HIV infected; HIV−, HIV uninfected; HU, Hounsfield Unit; CD206+, CD206 expressing; CD163+, CD163 expressing; MDMC, Mannosylated Dextran Molecular Construct

### FIGURES

FIG. 1. Shows an example of the molecular structure of ^{99m}Tc-tilmanocept.

FIGS. 2A-I. Show an aortic ^{99m}Tc-tilmanocept SPECT/CT in HIV+ and HIV− subjects.

FIGS. 3A-B. Show an example of an aortic volume and percent aortic volume with high-level ^{99m}Tc-tilmanocept uptake.

FIGS. 4A-B. Shows ^{99m}Tc-tilmanocept SPECT/CT uptake versus [18F]Na-PET/CT.

FIGS. 5A-F. Show results of Ex vivo experiments on tissue-banked aortic samples from individuals with and without HIV.

FIGS. 6A-C. Show a representative subject demonstrating ^{99m}Tc-tilmanocept uptake in the liver and kidney tissue.

FIGS. 7A-G. Show ^{99m}Tc-tilmanocept uptake on SPECT/CT of representative HIV+ subject in area of aortic plaque; CTA parameters; and relationship of high-level ^{99m}Tc-tilmanocept uptake to non-calcified aortic plaque volume.

FIGS. 8A-B. Show data related to the imaging acquisition parameters of ^{99m}Tc-Tilmanocept SPECT and 18F-NaF PET Injection.

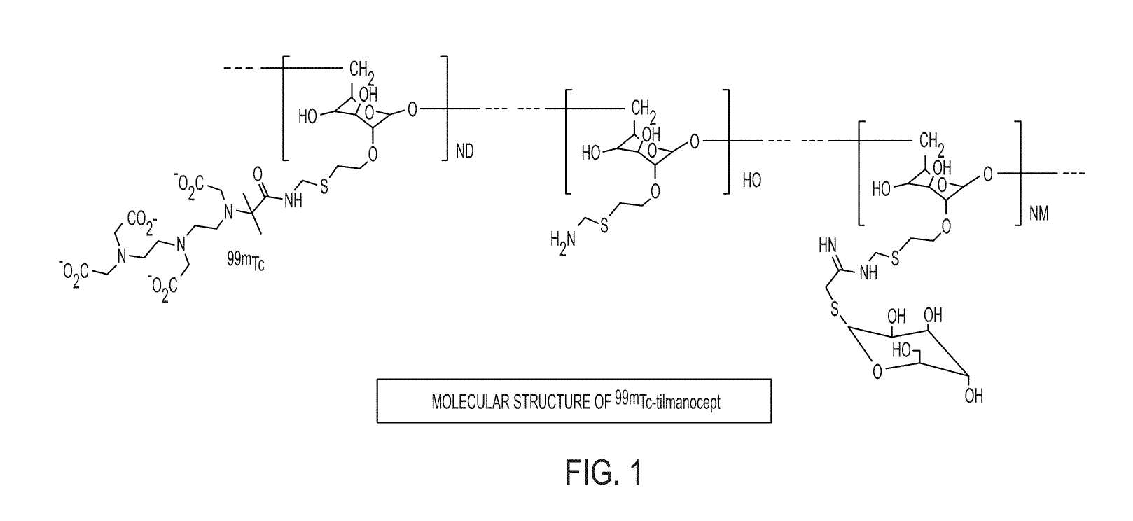

### FIG. 1. STRUCTURE OF MANNOSYLATED DEXTRAN MOLECULAR CONSTRUCTS (MDMCS) AS EXEMPLIFIED BY ^{99M}TC-TILMANOCEPT

^{99m}Tc-tilmanocept is an example of a mannosylated dextran molecular construct (MDMC). ^{99m}Tc-tilmanocept is comprised of a 10 kDₐ (10 kilo-Dalton) dextran backbone with multiple tethered moieties such as, but not limited to: diethylenetriamine-penta-acetic acid (DPTA, colored in blue) and mannose (colored in green). One skilled in the art would appreciate that dextran backbones of various sizes other than 10 kDₐ could be utilized to create MDMCs with similar performance attributes. ^{99m}Tc-tilmanocept avidly binds to the macrophage mannose receptor, CD206, via the mannose units. DPTA, a chelating agent, permits radiolabeling of tilmanocept with technectium (^{99m}Tc, a gamma-emitting metastable isotope). Radioactive labels other than ^{99m}Tc may be used and these would be ascertainable to one of skill in the art.

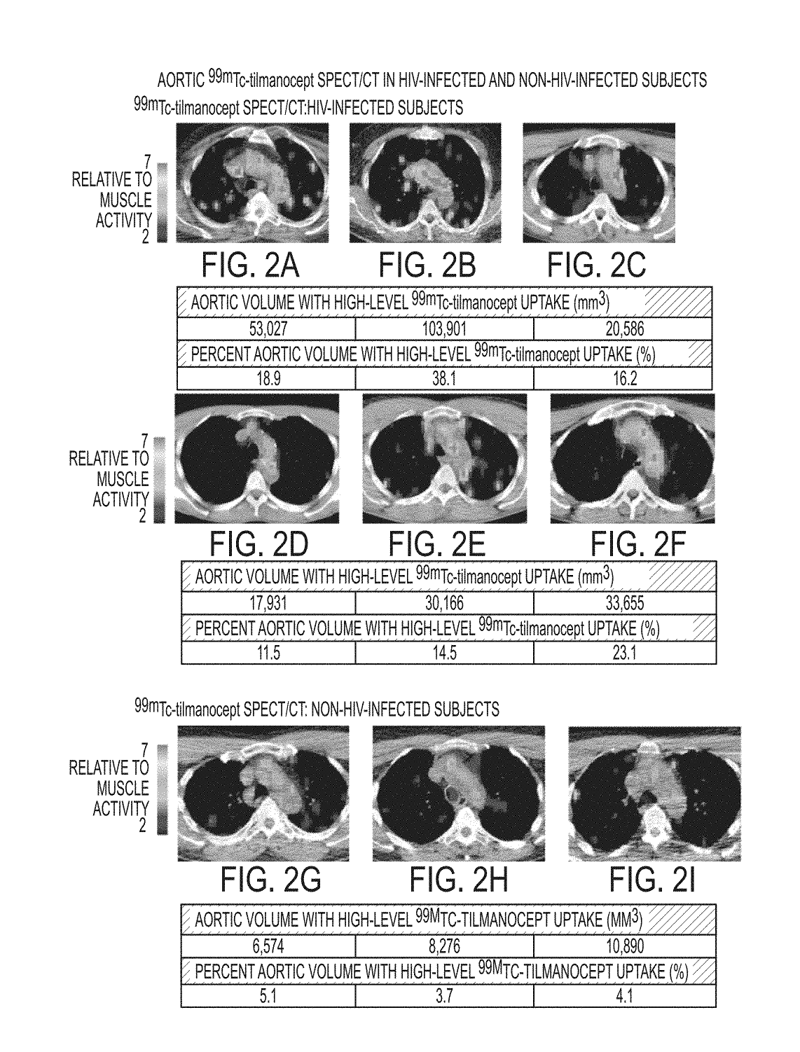

### FIGS. 2A-I. AORTIC ^{99M}TC-TILMANOCEPT SPECT/CT IN HIV+ AND HIV− SUBJECTS

FIGS. 2A-I show axial cross-sections of the aorta from ^{99m}Tc-tilmanocept SPECT/CT scans shown with HIV+ subjects at the top of the figure (FIGS. 2A-F) and HIV− subjects (FIGS. 2G-I) at the bottom of the figure. Aortic volume (mm³) and percent aortic volume with high-level ^{99m}Tc-tilmanocept uptake (greater than five times muscle ^{99m}Tc-tilmanocept uptake) are displayed for each subject below the respective axial cross-sectional images. Aortic ^{99m}Tc-tilmanocept uptake relative to muscle ^{99m}Tc-tilmanocept uptake is indicated based on the adjacent scale with red representing areas of high relative ^{99m}Tc-tilmanocept uptake and purple representing areas of low relative ^{99m}Tc-tilmanocept uptake.

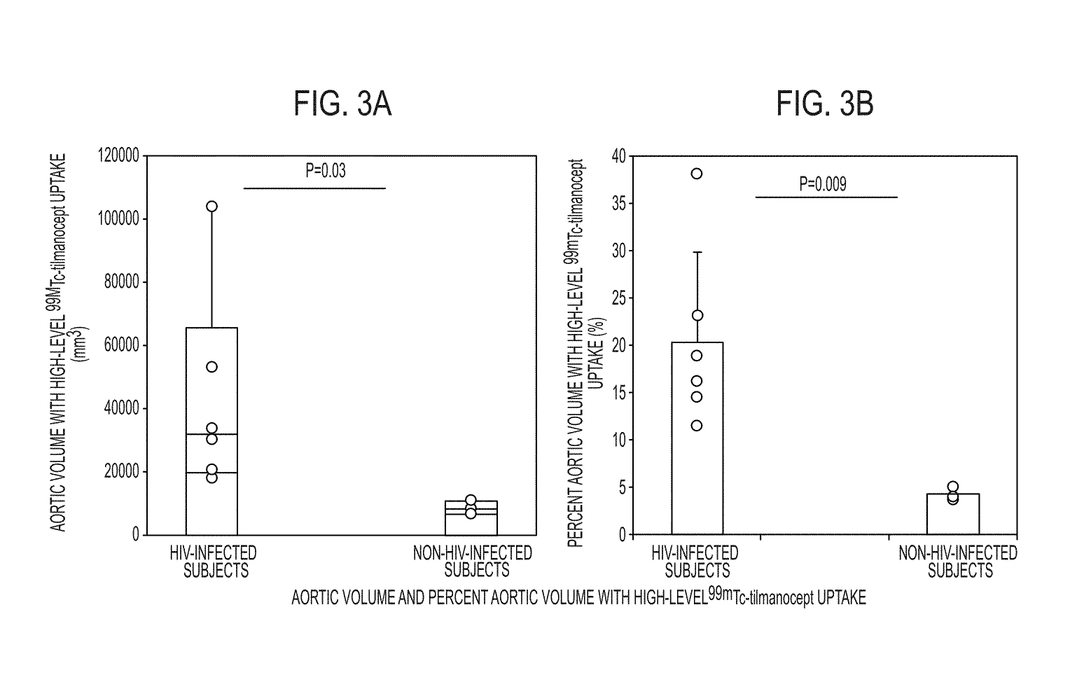

### FIGS. 3A-B. AORTIC VOLUME AND PERCENT AORTIC VOLUME WITH HIGH-LEVEL ^{99M}TC-TILMANOCEPT UPTAKE

FIG. 3A shows aortic volume with high-level ^{99m}Tc-tilmanocept uptake (with >5×^{99m}Tc-tilmanocept uptake in high-level ^{99m}Tc-tilmanocept that in muscle) among the HIV+ subjects compared to the HIV− subjects. Aortic volume with high-level ^{99m}Tc-tilmanocept uptake was significantly increased in the HIV+ subjects compared to the HIV− subjects [31,910 (19, 922, 65,745) vs. 8,276 (6,574, 10,890) mm³, P=0.03]. FIG. 3B shows the percent aortic volume with high-level ^{99m}Tc-tilmanocept uptake among the HIV+ subjects compared to the HIV− subjects. The percent aortic volume of high-level ^{99m}Tc-tilmanocept uptake was significantly increased among the HIV+ subjects compared to the HIV− subjects [20.4±9.5 vs. 4.3±0.70%, P=0.009].

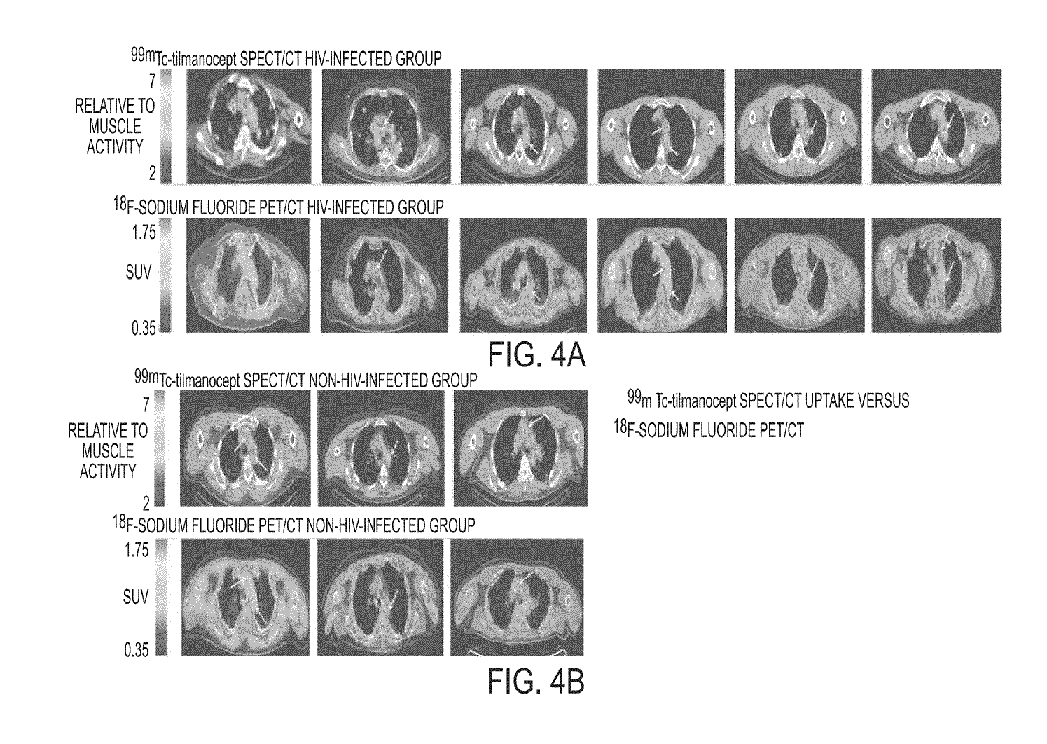

### FIGS. 4A-B. ^{99M}TC-TILMANOCEPT SPECT/CT UPTAKE VERSUS [18F]NA-PET/CT

Axial cross-sections of ^{99m}Tc-tilmanocept SPECT/CT (top row) and [18F]Na-PET/CT (bottom row) of HIV+ and HIV− subjects. Arrows in ^{99m}Tc-tilmanocept SPECT/CT demonstrate areas of high aortic ^{99m}Tc-tilmanocept uptake. Arrows in [18F]Na-PET/CT demonstrate the corresponding aortic sodium fluoride uptake in area highlighted on ^{99m}Tc-tilmanocept SPECT/CT scanning. In several of subjects, there were areas of high relative ^{99m}Tc-tilmanocept uptake on SPECT/CT with a corresponding low SUV on [18F]Na-PET/CT.

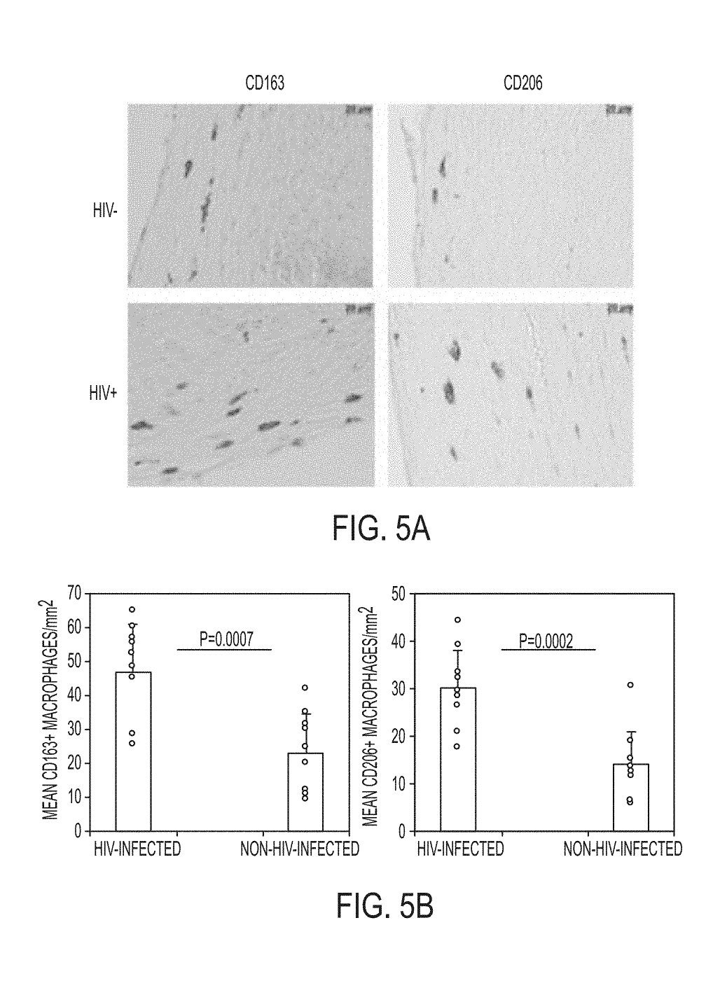

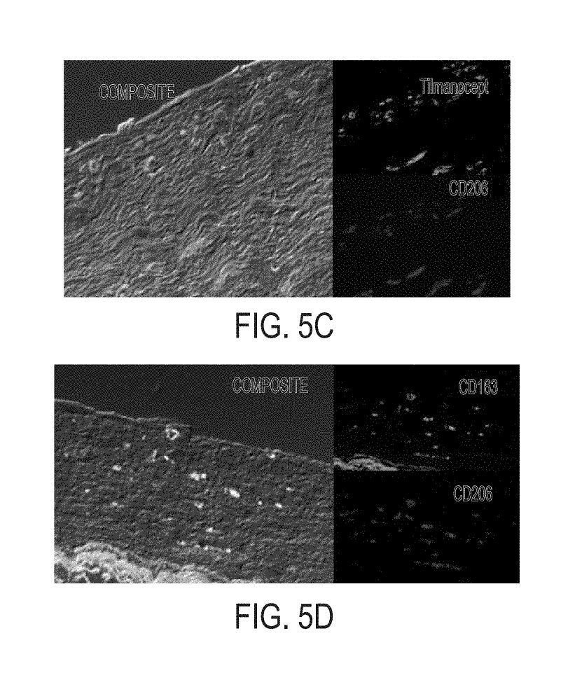

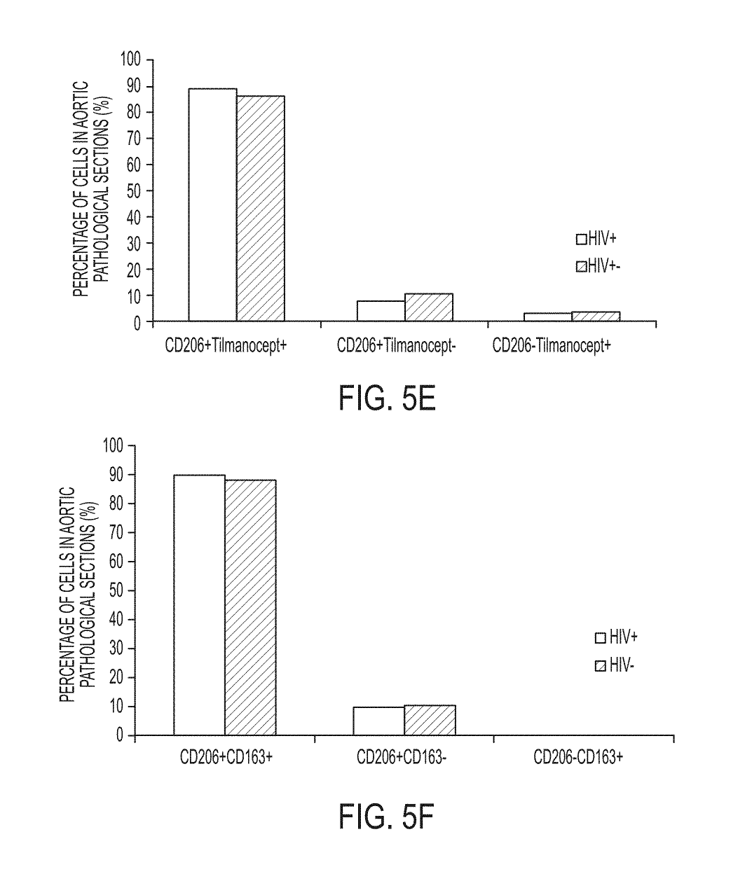

### FIGS. 5A-F. EX VIVO EXPERIMENTS ON TISSUE-BANKED AORTIC SAMPLES FROM INDIVIDUALS WITH AND WITHOUT HIV

FIGS. 5A-F show results of Ex vivo experiments on tissue-banked aortic samples from individuals with and without HIV. FIG. 5A shows the results of single label immunohistochemistry for CD206⁺ and CD163⁺ macrophages being performed on formalin-fixed paraffin embedded sections of aorta from 10 HIV-infected and 10 non-HIV-infected individuals. Sections were provided by the National NeuroAIDS Tissue Consortium (NNTC) and the National Disease Research Institute (NDRI). Representative sections of the aorta from HIV+ individuals and HIV− individuals with single staining of CD206⁺ and CD163⁺ macrophages are shown. FIG. 5B shows the average number of CD206+ and CD163+ macrophages/mm²± the standard deviation determined by counting the number of positive macrophages from twenty non-overlapping 200× fields of view (field area=0.148 mm²) per section. The mean number of CD206⁺ macrophages/mm² was significantly higher among HIV+ individuals vs. HIV− individuals (30.1±7.9 vs. 14. 2±7.0, P=0.0002). The mean number of CD163⁺ macrophages/mm² was also significantly higher for HIV+ individuals compared to HIV− individuals (46.7±14.2 vs. 22.9±11.6, P=0.0007). FIG. 5C shows double label immunofluorescence performed on formalin-fixed paraffin embedded sections of aorta from 10 HIV+ and 10 HIV− individuals using anti-CD206 antibodies and fluorescently labeled tilmanocept. There was a high and similar degree of co-localization of fluorescent tilmanocept and anti-CD206 in sections from both HIV+ and HIV− individuals (89.1±6.3% vs. 86.3±6.8%, respectively). The percentage of CD206⁺ tilmanocept⁻ macrophages was 7.8±7.0% in HIV+ individuals and 10.4±6.2% in HIV− individuals, whereas the percentage of CD206⁻ Tilmanocept⁺ macrophages was 3.1±1.8% in the HIV+ individuals and 3.3±2.1% in the HIV− individuals. FIG. 5D shows double label immunofluorescence performed on formalin-fixed paraffin embedded sections of aorta from 10 HIV+ and 10 HIV− individuals using antibodies against CD163 and CD206. There was a high degree of co-localization between CD163 and CD206 in sections from both HIV+ and HIV− individuals (90.0±6.3% vs. 88.2±4.5%, respectively). The percentage of CD163⁺CD206⁻ was 10.0±3.3% in HIV-infected individuals and 10.8±5.1% in non-HIV-infected individuals. There were no cells in sections of the aortas from any individuals that were CD163⁻CD206⁺.

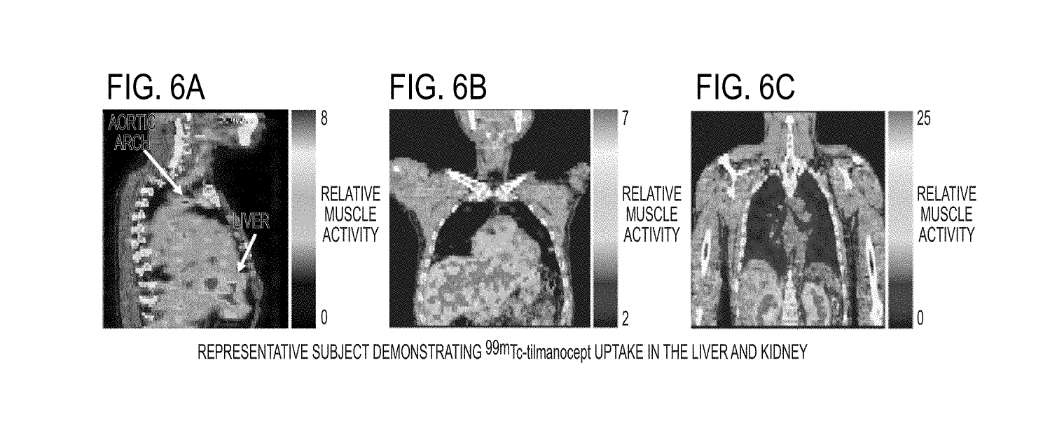

### FIGS. 6A-C. REPRESENTATIVE SUBJECT DEMONSTRATING ^{99M}TC-TILMANOCEPT UPTAKE IN THE LIVER AND KIDNEY

FIGS. 6A-C show a sagittal cross-section of a representative subject demonstrating intense ^{99m}Tc-tilmanocept uptake in the aorta and the liver. Arrows denote areas of high-level tilmanocept uptake in the aortic arch and liver respectively. FIG. 6b shows a coronal cross-section of representative subject demonstrating intense ^{99m}Tc-tilmanocept uptake in the liver relative to muscle activity. FIG. 6C Coronal cross-section of representative subject demonstrating intense ^{99m}Tc-tilmanocept uptake in the kidneys relative to muscle activity.

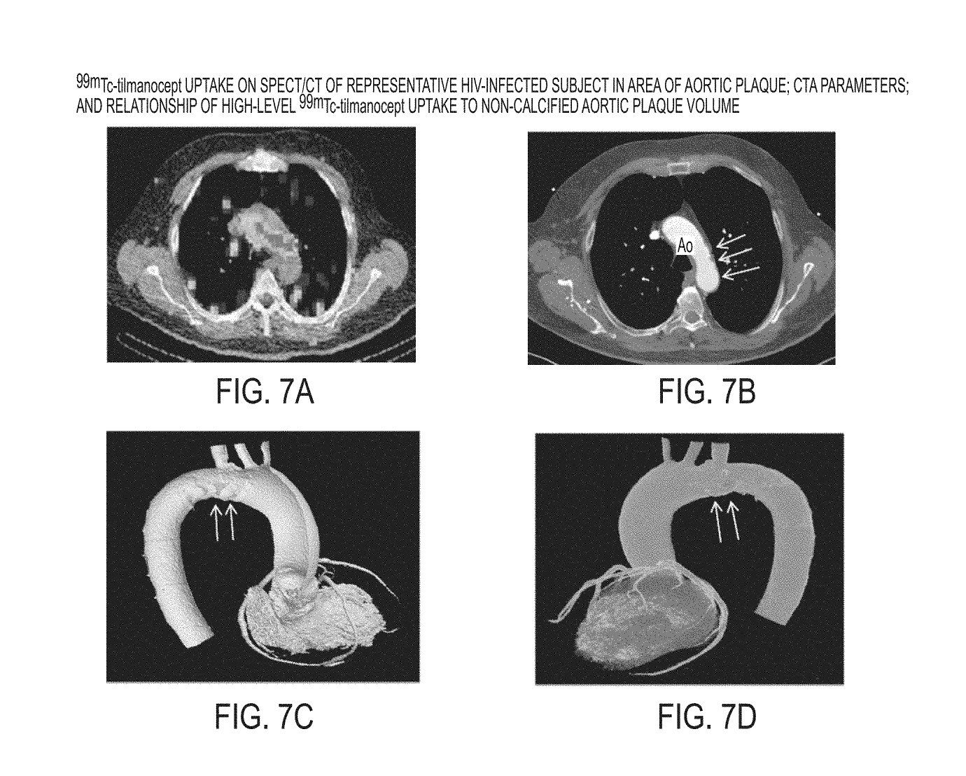

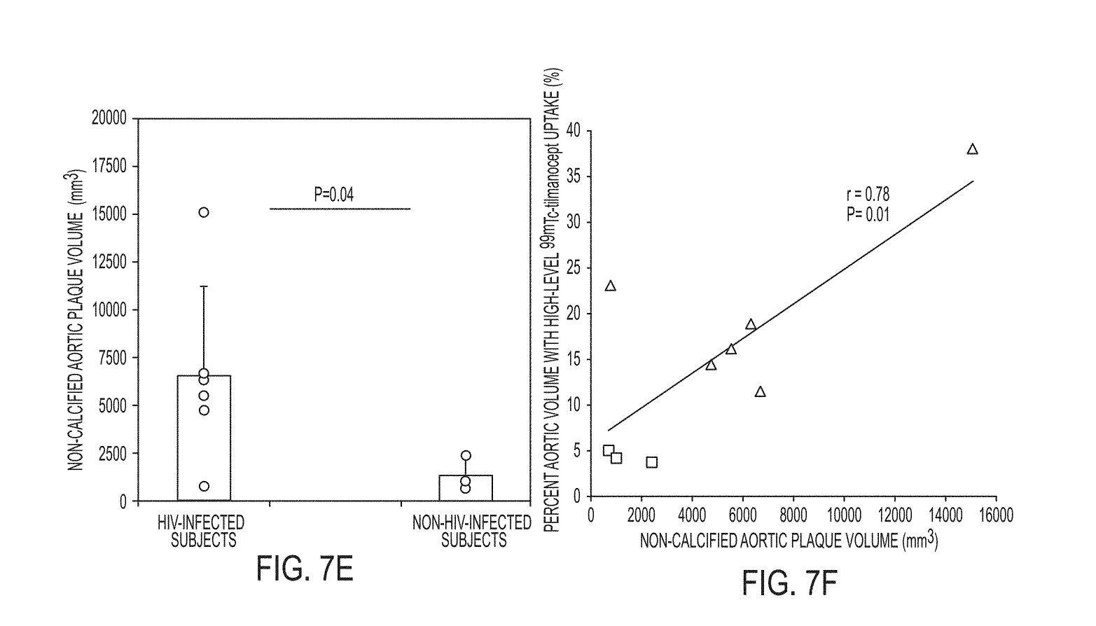

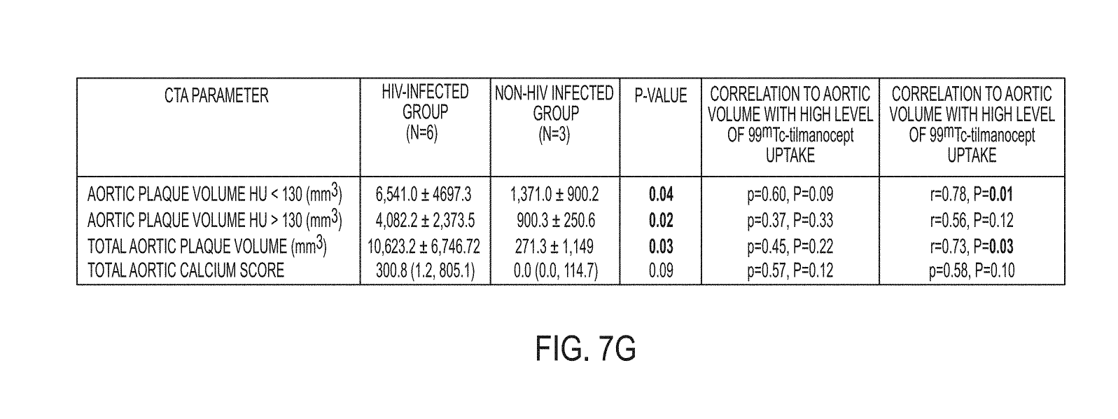

### FIGS. 7A-G. ^{99M}TC-TILMANOCEPT UPTAKE ON SPECT/CT OF REPRESENTATIVE HIV+ SUBJECT IN AREA OF AORTIC PLAQUE; CTA PARAMETERS; AND RELATIONSHIP OF HIGH-LEVEL ^{99M}TC-TILMANOCEPT UPTAKE TO NON-CALCIFIED AORTIC PLAQUE VOLUME

FIGS. 7A-G show ^{99m}Tc-tilmanocept uptake on SPECT/CT of representative HIV+ subject in area of aortic plaque; CTA parameters; and relationship of high-level ^{99m}Tc-tilmanocept uptake to non-calcified aortic plaque volume. FIG. 7A shows a ^{99m}Tc-tilmanocept SPECT/CT of a representative subject (corresponding with the subject of FIG. 2B demonstrating ^{99m}Tc-tilmanocept uptake in the aorta. FIG. 7B shows co-registered CTA of the same subject as in FIG. 7A demonstrating largely non-calcified aortic plaque (Hounsfield units <130, shown by arrows) in area of high-level ^{99m}Tc-tilmanocept uptake on SPECT/CT. FIG. 7C shows a three-dimensional volume rendering technique (VRT) reconstructions of the aorta from the representative subject in FIG. 7A demonstrating in the inner wall of the aorta with red arrows indicating areas of calcification within aortic plaque. FIG. 3D shows a three-dimensional volume rendering technique (VRT) reconstructions of the aorta from the representative subject in FIG. 7A demonstrating in the outer wall of the aorta with red arrows indicating areas of calcification within aortic plaque. FIG. 7E shows a comparison of non-calcified aortic plaque volume (Hounsfield units <130) among HIV+ subjects versus HIV− subjects. FIG. 7E demonstrates that non-calcified aortic plaque volume (Hounsfield units <130) was significantly higher among HIV+ subjects versus HIV− subjects (6,541.0±4,697.3 vs. 1,371.0±900.2, P=0.04). FIG. 7F shows a regression analysis relating percent aortic volume with high-level ^{99m}Tc-tilmanocept uptake on SPECT/CT and aortic plaque volume with HU<130 (non-calcified plaque). HIV+ subjects are represented as triangles (N=6) and HIV− control subjects are represented as squares (N=3). Pearson's correlation coefficient and corresponding r value for the relationship are given. There was a superior, unexpected, and significant relationship between percent aortic volume with high-level ^{99m}Tc-tilmanocept uptake on SPECT/CT and aortic plaque volume with HU <130 (non-calcified aortic plaque volume) (r=0.78, P=0.01). FIG. 7G shows the normally distributed data reported as mean±standard deviation and non-normally distributed data are reported as median (interquartile range). Bivariate analyses between (CTA) computed tomography angiography parameters and aortic volume and percent aortic volume with high-level ^{99m}Tc-tilmanocept uptake are shown as a Pearson's correlation coefficient (r) if both variables were normally distributed or Spearmans's rank correlation coefficient (p) if at least one variable was non-normally distributed as shown in FIG. 7G.

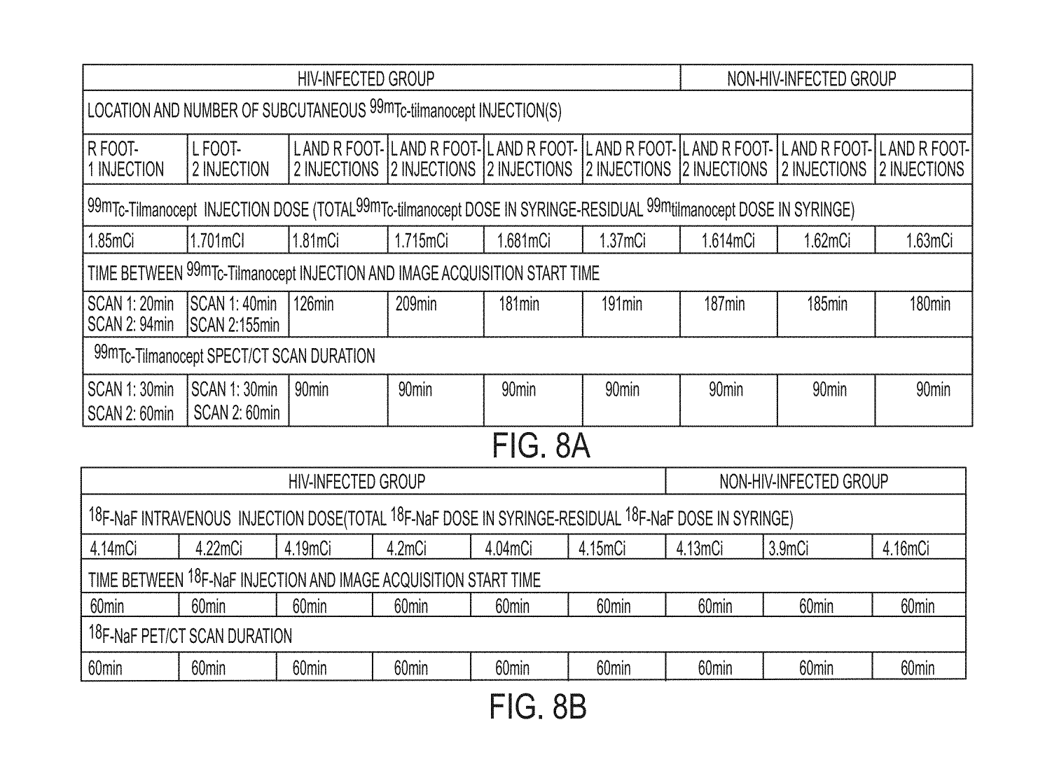

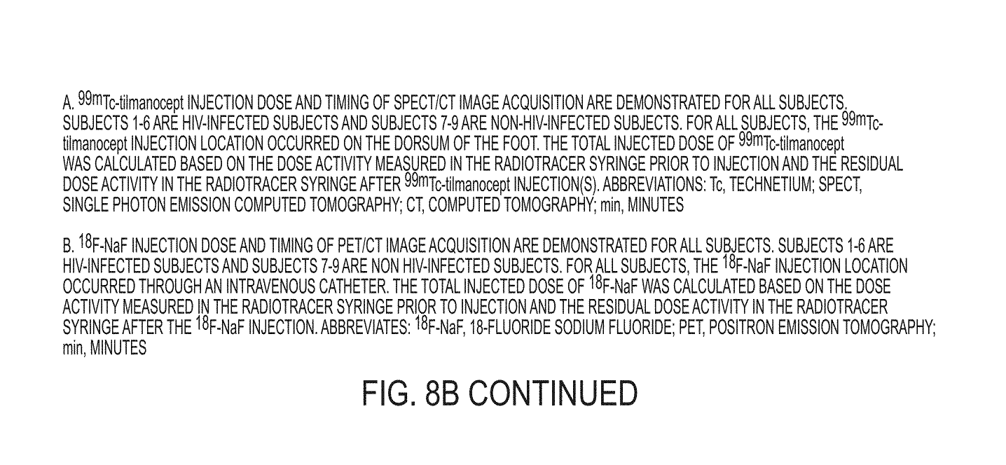

### FIGS. 8A-B. SHOW DATA RELATED TO THE IMAGING ACQUISITION PARAMETERS OF ^{99M}TC-TILMANOCEPT SPECT AND [18F]NA PET INJECTION

Certain embodiments comprise a compound comprising a dextran backbone having one or more CD206 targeting moieties and one or more diagnostic moieties attached thereto. Embodiments can comprise a compound according to claim 1, wherein the compound is a compound of Formula (II):

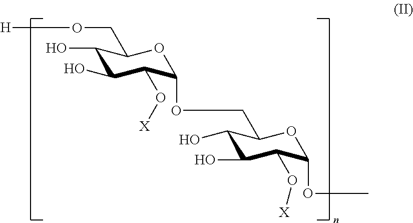

Wherein n is 1 or more and each X is independently H, L₁-A, or L₂-R; each L₁ and L₂ are independently linkers; each A independently comprises a detection moiety or H; each R independently comprises a CD206 targeting moiety or H; and n is an integer greater than zero; and wherein at least one R is a CD206 targeting moiety and at least one A is a diagnostic moiety. In some embodiments, at least one R is selected from the group consisting of mannose, fucose, and n-acetylglucosamine. In some embodiments, at least one A is a gamma-emitting agent. In some embodiments, at least one A can be selected from the group consisting of ^{99m}Tc, ¹¹¹In and ¹²³I. In some embodiments, the at least one A can be an isotope. In some embodiments, the at least one A is selected from the group consisting of ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr. In some embodiments, at least one L₁ a C₂₋₁₂ hydrocarbon chain optionally interrupted by up to three heteroatoms selected from the group consisting of O, S and N. In some embodiments, at least one L₁ comprises —(CH₂)ₚS(CH₂)_{q}NH—, wherein p and q are integers from 1 to 5. In some embodiments, at least one L₂ is a C₂₋₁₂ hydrocarbon chain optionally interrupted by up to nine heteroatoms selected from the group consisting of O, S and N. In some embodiments, at least one L₂ comprises —(CH₂)ₚS(CH₂)_{q}NH—, wherein p and q independently are integers from 1 to 5. In certain embodiments, the at least one A is a contrast agent suitable for computed tomographic (CT) imaging and the at least one A is selected from the group consisting of iodinated molecules, ytterbium and dysprosium.

Certain embodiments described herein can comprise a composition for imaging vascular inflammation, comprising: ^{99m}Tc-tilmanocept, wherein said composition is used for imaging vascular inflammation. Some embodiments comprise a composition for imaging vascular inflammation, comprising: ^{99m}Tc-tilmanocept, wherein said composition is used for imaging vascular inflammation.

Certain embodiments comprise a method of diagnosing vascular inflammation comprising: administering a compound to a subject comprising a dextran backbone having one or more CD206 targeting moieties and one or more diagnostic moieties attached thereto; and imaging said subject using single-photon emission computed technology (SPECT/CT) wherein an image comprises visual indications of uptake of said compound in the subject's vascular tissues. In some embodiments, the imaging of a subject can be done using planar gamma imaging wherein an image comprises visual indications of uptake of said compound in the subject's vascular tissues Some embodiments may comprise a method further comprising the step of quantifying the amount of non-calcified plaque in a subject. In certain embodiments, the method is non-invasive. In some embodiments the method further comprises quantifying the subject's atherosclerotic non-calcified plaque amounts. Certain embodiments comprise a method of anatomically locating non-calcified plaque in a subject. Certain embodiments comprise a method of diagnosing vascular inflammation comprising: administering a compound to a subject comprising a dextran backbone having one or more CD206 targeting moieties and one or more diagnostic moieties attached thereto.

Certain embodiments described herein comprise a composition for measuring atherosclerotic non-calcified plaque, comprising: ^{99m}Tc-tilmanocept, wherein said composition is used for imaging atherosclerotic plaque. Certain embodiments can comprise a composition for non-invasive imaging of atherosclerotic plaque, comprising: ^{99m}Tc-tilmanocept, wherein said composition is used for imaging atherosclerotic plaque. In some embodiments, the subject is infected with human immunodeficiency virus (HIV).

Certain embodiments can comprise a composition for non-invasively measuring atherosclerotic plaque, including non-calcified atherosclerotic plaque. Certain embodiments can comprise a composition comprising a diagnostic moiety for quantifying non-calcified atherosclerotic plaque. In some embodiments, the composition is non-invasive and can be used to quantify a subject's non-calcified plaque amounts.

Some embodiments can comprise a composition for measuring atherosclerotic plaque, comprising: ^{99m}Tc-tilmanocept (or other composition as described herein), wherein said composition is used for imaging atherosclerotic plaque and measuring atherosclerotic plaque in a subject. Certain embodiments can comprise a composition for non-invasive imaging of atherosclerotic plaque, comprising: ^{99m}Tc-tilmanocept, wherein said composition is used for imaging atherosclerotic plaque. In some embodiments, the subject is infected with human immunodeficiency virus (HIV).

Certain embodiments can comprise a composition for non-invasively measuring non-calcified atherosclerotic plaque. Some embodiments can comprise a composition for quantifying atherosclerotic plaque in a subject and the composition can be non-invasive.

Some embodiments comprise a mannosylated dextran molecular construct comprised of glucose moieties comprising: a backbone comprised of dextran, at least one leash attached to the glucose moieties of the dextran backbone, at least one mannose sugars attached to a portion of the at least one leash; and one or more detection moieties attached to the at least one leash. In certain embodiments, the at least one leash is not occupied by any mannose moieties. In some embodiments, the at least one other sugar moiety can be added to the at least one leash, wherein the at least one other sugar is not occupied by either mannose moiety or a detection moiety.

Some embodiments can comprise a method of diagnosing atherosclerotic inflammation comprising administering any of the compositions described herein. Some embodiments can comprise a method of identifying and/or quantifying non-calcified plaque in a subject comprising administering any of the compositions described herein. Certain embodiments can comprise a method of quantifying atherosclerotic plaque in a subject comprising administering any of the compositions described herein. Certain embodiments can comprise a method of quantifying the amount of non-calcified atherosclerotic plaque in a subject comprising administering any of the compositions described herein. Some methods described herein can include the step of determining a subject's likelihood of developing cardiovascular diseases, such as those described herein.

Certain embodiments can comprise a composition for imaging atherosclerotic plaque comprising: a diagnostic moiety, wherein said composition is used for imaging atherosclerotic plaque.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Perkin Elmer Corporation, U.S.A.).

As used in the specification, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification to parts by weight of a particular element or component in a composition denotes the weight relationship between the element or component and any other elements or components in the composition or article for which a part by weight is expressed, unless expressly described otherwise. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the identification can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent.

The term “contacting” as used herein refers to bringing a disclosed compound and a cell, a target receptor (e.g. CD206, or other receptor), or other biological entity together in such a manner that the compound can affect the activity of the target, either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, The specific effective amount for any particular subject will depend upon a variety of factors including the disorder being diagnosed and the severity of the disorder; the specific composition employed; the age, body weight, general health, sex and diet of the patient; the time of administration; the route of administration; the rate of excretion of the specific compound employed; the duration of the diagnosis; drugs used in combination or coincidental with the specific compound employed and like factors well known in the medical arts. For example, it is well within the skill of the art to start doses of a compound at levels lower than those required to achieve the desired diagnostic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein the term “non-invasive” can refer to techniques that do not include the insertion or introduction of any instruments into a subject. For instance, administration of a diagnostic agent with a diagnostic moiety could be injected into a subject as described herein and then imaging, measuring, analyzing techniques described herein can be used to exercise the methods described herein. The term “non-invasive” will be clear to the skilled artisan when viewing the term in the context in which it used herein.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

“Alkyl” refers to a saturated aliphatic hydrocarbon including straight chain and branched chain groups. “Alkyl” may be exemplified by groups such as methyl, ethyl, n-propyl, isopropyl, n-butyl and the like. Alkyl groups may be substituted or unsubstituted. More than one substituent may be present. Substituents may also be themselves substituted. When substituted, the substituent group is preferably but not limited to C₁-C₄ alkyl, aryl, heteroaryl, amino, imino, cyano, halogen, alkoxy or hydroxyl. “C₁-C₄ alkyl” refers to alkyl groups containing one to four carbon atoms.

“Alkenyl” refers to an unsaturated aliphatic hydrocarbon moiety including straight chain and branched chain groups. Alkenyl moieties must contain at least one alkene. “Alkenyl” may be exemplified by groups such as ethenyl, n-propenyl, isopropenyl, n-butenyl and the like. Alkenyl groups may be substituted or unsubstituted. More than one substituent may be present. When substituted, the substituent group is preferably alkyl, halogen or alkoxy. Substituents may also be themselves substituted. Substituents can be placed on the alkene itself and also on the adjacent member atoms or the alkenyl moiety. “C₂-C₄ alkenyl” refers to alkenyl groups containing two to four carbon atoms.

“Alkynyl” refers to an unsaturated aliphatic hydrocarbon moiety including straight chain and branched chain groups. Alkynyl moieties must contain at least one alkyne. “Alkynyl” may be exemplified by groups such as ethynyl, propynyl, n-butynyl and the like. Alkynyl groups may be substituted or unsubstituted. More than one substituent may be present. When substituted, the substituent group is preferably alkyl, amino, cyano, halogen, alkoxyl or hydroxyl. Substituents may also be themselves substituted. Substituents are not on the alkyne itself but on the adjacent member atoms of the alkynyl moiety. “C₂-C₄ alkynyl” refers to alkynyl groups containing two to four carbon atoms.

“Acyl” or “carbonyl” refers to the group —C(O)R wherein R is alkyl; alkenyl; alkynyl, aryl, heteroaryl, carbocyclic, heterocarbocyclic; C₁-C₄ alkyl aryl or C₁-C₄ alkyl heteroaryl. C₁-C₄ alkylcarbonyl refers to a group wherein the carbonyl moiety is preceded by an alkyl chain of 1-4 carbon atoms.

“Alkoxy” refers to the group —O—R wherein R is acyl, alkyl alkenyl, alkyl alkynyl, aryl, carbocyclic; heterocarbocyclic; heteroaryl, C₁-C₄ alkyl aryl or C₁-C₄ alkyl heteroaryl.

“Amino” refers to the group —NR′R′ wherein each R′ is, independently, hydrogen, amino, hydroxyl, alkoxyl, alkyl, aryl, cycloalkyl, heterocycloalkyl, heteroaryl, C₁-C₄ alkyl aryl or C₁-C₄ alkyl heteroaryl. The two R′ groups may themselves be linked to form a ring. The R′ groups may themselves be further substituted, in which case the group also known as guanidinyl is specifically contemplated under the term ‘amino”.

“Aryl” refers to an aromatic carbocyclic group. “Aryl” may be exemplified by phenyl. The aryl group may be substituted or unsubstituted. More than one substituent may be present. Substituents may also be themselves substituted. When substituted, the substituent group is preferably but not limited to heteroaryl, acyl, carboxyl, carbonylamino, nitro, amino, cyano, halogen, or hydroxyl.

“Carboxyl” refers to the group —C(═O)O—C₁-C₄ alkyl.

“Carbonyl” refers to the group —C(O)R wherein each R is, independently, hydrogen, alkyl, aryl, cycloalkyl; heterocycloalkyl, heteroaryl, C₁-C₄ alkyl aryl or C₁-C₄ alkyl heteroaryl.

“Carbonylamino” refers to the group —C(O)NR′R′ wherein each R′ is, independently, hydrogen, alkyl, aryl, cycloalkyl, heterocycloalkyl, heteroaryl, C₁-C₄ alkyl aryl or C₁-C₄ alkyl heteroaryl. The two R′ groups may themselves be linked to form a ring.

“C₁-C₄ alkyl aryl” refers to C₁-C₄ alkyl groups having an aryl substituent such that the aryl substituent is bonded through an alkyl group. “C₁-C₄ alkyl aryl” may be exemplified by benzyl.

“C₁-C₄ alkyl heteroaryl” refers to C₁-C₄ alkyl groups having a heteroaryl substituent such that the heteroaryl substituent is bonded through an alkyl group.

“Carbocyclic group” or “cycloalkyl” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, preferably 4 to 7 carbon atoms, and more preferably 5 to 6 carbon atoms in the ring. Bicyclic carbocyclic groups contain 8 to 12 carbon atoms, preferably 9 to 10 carbon atoms in the ring. Carbocyclic groups may be substituted or unsubstituted. More than one substituent may be present. Substituents may also themselves be substituted. Preferred carbocyclic groups include cyclopropyl, cyclobutyl, cyclopentyl, cyclohexyl, cyclohexenyl, and cycloheptyl. More preferred carbocyclic groups include cyclopropyl and cyclobutyl. The most preferred carbocyclic group is cyclopropyl. Carbocyclic groups are not aromatic.

As also used herein, the term “diagnosing” means determining the presence or absence of a medical condition, as well as determining or confirming the status of a previously confirmed medical condition in a patient. For example, in the case of cancer, the term diagnosing encompasses determining the presence or absence of cancer, the stage of cancer, and/or the detection of the presence, absence, or stage of a precancerous condition in a patient. Determining the status of a previously confirmed medical condition also includes determining the progress, lack of progress, decline or remission of a medical condition (e.g., a macrophage-related disorder).

“Halogen” refers to fluoro, chloro, bromo or iodo moieties. Preferably, the halogen is fluoro, chloro, or bromo.

“Heteroaryl” or “heteroaromatic” refers to a monocyclic or bicyclic aromatic carbocyclic radical having one or more heteroatoms in the carbocyclic ring. Heteroaryl may be substituted or unsubstituted. More than one substituent may be present. When substituted, the substituents may themselves be substituted. Preferred but non limiting substituents are aryl, C₁-C₄ alkylaryl, amino, halogen, hydroxy, cyano, nitro, carboxyl, carbonylamino, or C₁-C₄ alkyl. Preferred heteroaromatic groups include tetrazoyl, triazolyl, thienyl, thiazolyl, purinyl, pyrimidyl, pyridyl, and furanyl. More preferred heteroaromatic groups include benzothiofuranyl; thienyl, furanyl, tetrazoyl, triazolyl, and pyridyl.

“Heteroatom” means an atom other than carbon in the ring of a heterocyclic group or a heteroaromatic group or the chain of a heterogeneous group. Preferably, heteroatoms are selected from the group consisting of nitrogen, sulfur, and oxygen atoms. Groups containing more than one heteroatom may contain different heteroatoms.

“Heterocarbocyclic group” or “heterocycloalkyl” or “heterocyclic” means a monovalent saturated or unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, preferably 4 to 7 carbon atoms, and more preferably 5 to 6 carbon atoms in the ring. Bicyclic heterocarbocyclic groups contain 8 to 12 carbon atoms, preferably 9 to 10 carbon atoms in the ring. Heterocarbocyclic groups may be substituted or unsubstituted. More than one substituent may be present. Substituents may also be themselves substituted. Preferred heterocarbocyclic groups include epoxy, tetrahydrofuranyl, azacyclopentyl, azacyclohexyl, piperidyl, and homopiperidyl. More preferred heterocarbocyclic groups include piperidyl, and homopiperidyl. The most preferred heterocarbocyclic group is piperidyl. Heterocarbocyclic groups are not aromatic.

“Hydroxy” or “hydroxyl” means a chemical entity that consists of —OH. Alcohols contain hydroxy groups. Hydroxy groups may be free or protected. An alternative name for hydroxy is hydroxyl.

“Leash/leashes” and “linker/linkers” may be used interchangeably herein. The term “leash” or “leashes” can often be used to refer to attachment moiety used for a targeting moiety, such as mannose. The term “linker” or “linkers” can be used to refer to the attachment moiety used for a diagnostic moiety that may incorporate additional properties related to the chemistry of the linker and diagnostic moiety and the delivery of the said agent. Although these terms can be used interchangeably herein, their meaning will be clear to the skilled artisan in view of the context with which it is used.

“Member atom” means a carbon, nitrogen, oxygen or sulfur atom. Member atoms may be substituted up to their normal valence. If substitution is not specified the substituents required for valency are hydrogen.

“Ring” means a collection of member atoms that are cyclic. Rings may be carbocyclic, aromatic, or heterocyclic or heteroaromatic, and may be substituted or unsubstituted, and may be saturated or unsaturated. More than one substituent may be present. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Thioalkyl” refers to the group —S— alkyl.

“Tilmanocept” can refer to a non-radiolabeled precursor of the LYMPHOSEEK® diagnostic agent. Compositions described herein may be a mannosylaminodextran. They can have a dextran backbone to which a plurality of amino-terminated linkers (—O(CH₂)₃S(CH₂)₂NH₂) are attached to the core glucose elements. In addition, mannose moieties can be conjugated to amino groups of a number of the linkers, and the chelator diethylenetriamine pentaacetic acid (DTPA) can be conjugated to the amino group of other linkers not containing the mannose. Compositions described herein can have a dextran backbone, in which a plurality of glucose residues comprise an amino-terminated linker:

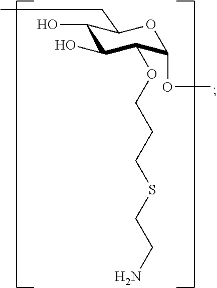

The mannose moieties can be conjugated to the amino groups of the linker via an amidine linker:

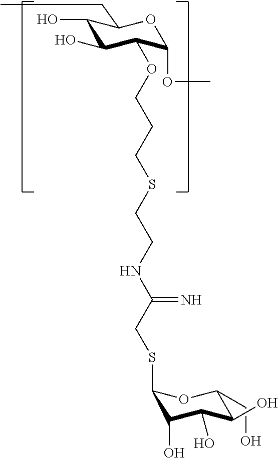

The chelator diethylenetriamine pentaacetic acid (DTPA) can be conjugated to the amino groups the linker via an amide linker:

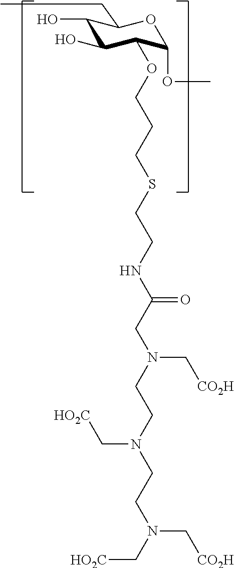

As described in the prescribing information approved for LYMPHOSEEK® in the United States, tilmanocept has the chemical name dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, has the following molecular formula: [C₆H₁₀O₅]ₙ●(C₁₉H₂₈N₄O₉S^{99m}Tc)_{b}●(C₁₃H₂₄N₂O₅S₂)_{c}●(C₅H₁₁NS)ₐ, and contains 3-8 conjugated DTPA molecules; 12-20 conjugated mannose molecules; and 0-17 amine side chains remaining free. Tilmanocept has the following general structure:

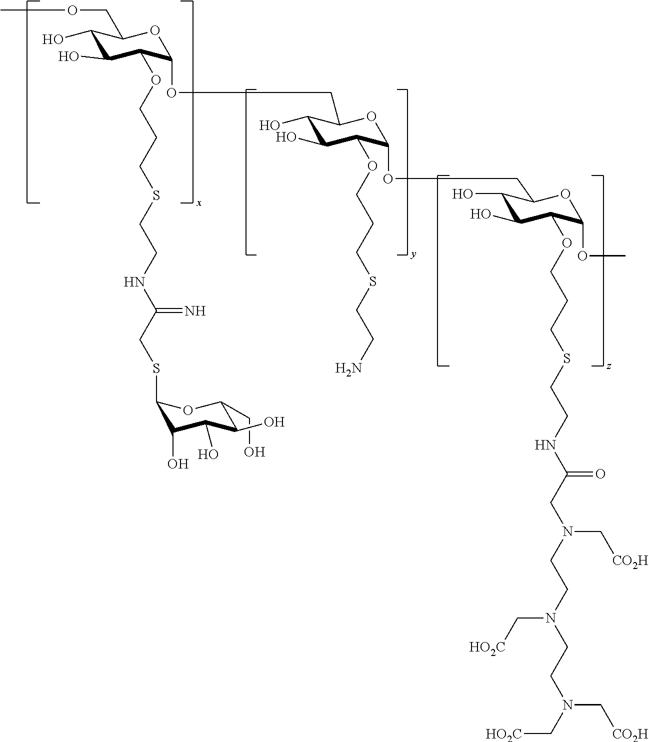

Certain of the glucose moieties may have no attached amino-terminated linker.

“Sulfonyl” refers to the —S(O)₂R′ group wherein R′ is alkoxy, alkyl, aryl, carbocyclic, heterocarbocyclic; heteroaryl, C₁-C₄ alkyl aryl or C₁-C₄ alkyl heteroaryl.

“Sulfonylamino” refers to the —S(O)₂NR′R′ group wherein each R′ is independently alkyl, aryl, heteroaryl, C₁-C₄ alkyl aryl or C₁-C₄ alkyl heteroaryl.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein can comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹³N, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes may be used for their ease of preparation and detectability. Further, substitution with heavier isotopes such as deuterium, i.e., ²H, can afford certain advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental Volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Compounds

Embodiments of the present invention can employ a carrier construct comprising a polymeric (e.g., carbohydrate) backbone that can comprise a CD206 targeting moiety attached thereto (e.g., mannose) to deliver one or more active pharmaceutical ingredients. Examples of such constructs include mannosylamino dextrans (MAD), which can comprise a dextran backbone having conjugated to glucose residues of the backbone mannose molecules and having conjugated to other glucose residues of the backbone an active pharmaceutical ingredient. Tilmanocept is a specific example of a MAD. A tilmanocept derivative that is tilmanocept without DTPA conjugated thereto is a further example of a MAD (sometimes referred to as m-tilmanocept).

In some embodiments, the present invention provides a compound comprising a dextran-based moiety or backbone having one or more CD206 targeting moieties attached thereto. The dextran-based moiety generally comprises a dextran backbone similar to that described in U.S. Pat. No. 6,409,990 (the '990 patent), which is incorporated herein by reference in its entirety. Thus, the backbone comprises a plurality of glucose moieties (i.e., residues) primarily linked by α-1,6 glycosidic bonds. Other linkages such as α-1,4 and/or α-1,3 bonds may also be present. In some embodiments, not every backbone moiety is substituted. In some embodiments, CD206 targeting moieties are attached to between about 10% and about 50% of the glucose residues of the dextran backbone, or between about 20% and about 45% of the glucose residues, or between about 25% and about 40% of the glucose residues. In some embodiments, every three glucose residues may be substituted. In some embodiments, every four glucose residues may be substituted. In some embodiments, every five glucose residues may be substituted. Some embodiments may comprise one mannose positioned on every third glucose residue. Some embodiments may comprise one mannose positioned on every fourth glucose residue. Some embodiments may comprise one mannose positioned on every fifth glucose residue. In some embodiments, the dextran-based moiety is about 50-100 kilodaltons (kDa). The dextran-based moiety may be at least about 50 kDa, at least about 60 kDa, at least about 70 kDa, at least about 80 kDa, or at least about 90 kDa. The dextran-based moiety may be less than about 100 kDa, less than about 90 kDa, less than about 80 kDa, less than about 70 kDa, or less than about 60 kDa. In some embodiments, the dextran backbone has a molecular weight (MW) of between about 1 and about 50 kDa, while in other embodiments the dextran backbone can have a MW of between about 5 and about 25 kDa. In embodiments, the dextran backbone can have a MW of between about 8 and about 15 kDa, such as about 10 kDa. While in other embodiments the dextran backbone can have a MW of between about 1 and about 5 kDa, such as about 2 kDa. Certain embodiments of compositions can comprise a backbone that is between about 1 to about 5 kDa, about 1 to about 10 kDa, about 1 to about 15 kDa, about 5 to about 12 kDa, about 5 to about 10 kDa, and ranges therebetween. In some embodiments, a composition may comprise between about 3 to about 7 mannose molecules, about 5 to about 10 mannose molecules, about 10 to about 15 mannose molecules, about 15 to about 20 mannose molecules, about 16 to about 17 mannose molecules, and ranges therebetween. In some embodiments, a backbone may be about 1 to about 3 kDa and may further comprise about 3 to about 7 mannose molecules. In some embodiments, a backbone may be about 10 kDa and may further comprise about 15 to about to about 20, or about 16 to about 17 mannose molecules. An embodiment may comprise a backbone that is about 10 kDa and further comprise about 16 to about 17 mannose molecules. Such a configuration has unexpectedly superior and improved solubility, improved clarity, improved injectability and distribution.

Some embodiments may comprise a backbone that is not a dextran backbone. Some embodiments may have a monosaccharide-based backbone that does not comprise dextran. The backbone of a carbohydrate-based carrier molecules described herein can comprise a glycan other than dextran, wherein the glycan comprises a plurality of monosaccharide residues (i.e., sugar residues or modified sugar residues). In certain embodiments, the glycan backbone has sufficient monosaccharide residues, as well as optional groups such as one or more amino acids, polypeptides and/or lipids, to provide a MW of about 1 to about 50 kDa. The glycan can comprise oligosaccharides or polysaccharides. As would be appreciated by the skilled artisan when considering the disclosure contained herein, when referring to a “dextran” backbone, other monosaccharide residues may be considered to be substituted in compounds described herein. Additional descriptions of carbohydrate-backbone-based carrier molecules used for targeting CD206 are described in PCT application No. US/2017/055211, which is herein incorporated by reference in its entirety.

In any of the embodiments where the backbone is conjugated with one or more primary carbohydrates (monosaccharides), such carbohydrates can comprise any of a variety of sugar and modified sugar residues (e.g., sulfated, brominated, or nitrogenated sugar residues), including one or more of: fucose, arabinose, allose, altrose, glucose, galactose, glucose, galactosamine, n-acetylgalactosamine, hammelose, lyxose, levoglucosenone, mannose, mannitol, mannosamine, n-acetylmannosamine, ribose, rhamnose, threose, talose, xylose and combinations of two or more of the foregoing. In certain embodiments, a backbone of compositions herein may comprise a carbohydrate moiety that does not comprise glucose and may be any suitable polymer. These moieties may include, for example but without limitation, fucose, n-acetylglucoseamine, n-acetylgalactoseamine, galactose, neuraminate, and the like. The backbone may be heterogeneous, containing more than one species of sugar and/or carbohydrate.

The carrier molecules used in the compositions, kits and diagnostic methods described herein are used to deliver a detectable moiety. The carrier molecules include one or more features which allow a detectable moiety to be attached to the molecule, either directly or indirectly (e.g., using a leash). In some embodiments, the carbohydrate-based backbone has a MW of between about 1 and about 50 kDa, while in other embodiments the carbohydrate-based backbone has a MW of between about 5 and about 25 kDa. In still other embodiments, the carbohydrate-based backbone has a MW of between about 8 and about 15 kDa, such as about 10 kDa. While in other embodiments the carbohydrate-based backbone has a MW of between about 1 and about 5 kDa, such as about 2 kDa. The MW of the carbohydrate-based backbone may be selected based upon the inflammasome-mediated disorder. In addition, unlike the dextran backbone of the '990 patent, the carbohydrate-based backbones described herein do not necessarily need to be crosslink-free, and larger MW backbones (>50 kDa) may be employed in some instances.

Any of a variety of detectable moieties can be attached to the carrier molecule, directly or indirectly, for a variety of purposes. As used herein, the term “detectable moiety” or “diagnostic moiety” (which these terms may be used interchangeably) means an atom, isotope, or chemical structure which is: (1) capable of attachment to the carrier molecule; (2) non-toxic to humans; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means as well as by the measurement processes described herein.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents. By way of specific example, suitable diagnostic moieties include, but are not limited to:
contrast agents suitable for magnetic resonance imaging (MRI), such as gadolinium (Gd³⁺), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr; or other chelateable isotope(s);
gamma-emitting agents suitable for single-photon emission computed tomography (SPECT), such as ^{99m}Tc, ¹¹¹In, and ¹²³I.
dyes and fluorescent agents suitable for optical imaging
agents suitable for positron emission tomography (PET) such as ¹⁸F.

A diagnostic moiety can be attached to the carrier molecule in a variety of ways, such as by direct attachment or using a chelator attached to a carrier molecule. In some embodiments, diagnostic moieties can be attached using leashes attached to a carrier backbone. Thereafter, and as described in the ties as by direct attack can be conjugated to an amino group of one or more leashes and can be used to bind the diagnostic moiety thereto. It should be noted that in some instances, glucose moieties may have no attached aminothiol leash. Certain embodiments may include a single type of diagnostic moiety or a mixture of different diagnostic moieties. For example, an embodiment of a compound disclosed herein may comprise a contrast agent suitable for MRI and a radioisotope suitable for scintigraphic imaging, and further combinations of the diagnostic moieties described herein.

One or more detectable moieties can be attached to the one or more leashes using a suitable chelator. Suitable chelators include ones known to those skilled in the art or hereafter developed, such as, for example but without limitation, tetraazacyclododecanetetraacetic acid (DOTA), mercaptoacetylglycylglycyl-glycine (MAG3), diethylenetriamine pentaacetic acid (DTPA), dimercaptosuccinic acid, diphenylehtylene diamine, porphyrin, iminodiacetic acid, and ethylenediaminetetraacetic acid (EDTA).

Certain embodiments of compositions can comprise a backbone that is between about 1 to about 5 kDa, about 1 to about 10 kDa, about 1 to about 15 kDa, about 5 to about 12 kDa, about 5 to about 10 kDa, and ranges there between. In some embodiments, a composition may comprise between about 2 to about 7 mannose molecules, about 5 to about 10 mannose molecules, about 10 to about 15 mannose molecules, about 15 to about 28 mannose molecules, about 16 to about 17 mannose molecules, and ranges there between. In some embodiments, a backbone may be about 1 to about 3 kDa and may further comprise about 3 to about 7 mannose molecules. In some embodiments, a backbone may be about 10 kDa and may further comprise about 15 to about to about 20, or about 16 to about 17 mannose molecules.

In some embodiments, the CD206 targeting moiety is selected from, but not limited to, mannose, fucose, fucoid, galactose, n-acetylgalactosamine, and n-acetylglucosamine and combinations of these. In some embodiments, the targeting moieties are attached to between about 10% and about 50% of the glucose residues of the dextran backbone, or between about 20% and about 45% of the glucose residues, or between about 25% and about 40% of the glucose residues. (It should be noted that the MWs referenced herein, as well as the number and degree of conjugation of receptor substrates, linkers, and diagnostic moieties attached to the dextran backbone refer to average amounts for a given quantity of carrier molecules, since the synthesis techniques will result in some variability.)

In some embodiments, the one or more CD206 targeting moieties and one or more detection labels are attached to the dextran-based moiety through a linker. The linker may be attached at from about 50% to about 100% of the backbone moieties or about 70% to about 90%. In embodiments with multiple linkers, the linkers may be the same or different. In some embodiments, the linker is an amino-terminated linker. In some embodiments, the linkers may comprise —O(CH₂)₃S(CH₂)₂NH—. In some embodiments, the linker may be a chain of from 1 to 20 member atoms selected from carbon, oxygen, sulfur, nitrogen and phosphorus. The linker may be a straight chain or branched. The linker may also be substituted with one or more substituents including, but not limited to, halo groups, perfluoroalkyl groups, perfluoroalkoxy groups, alkyl groups, such C₁₋₄ alkyl, alkenyl groups, such as C₁₋₄ alkenyl, alkynyl groups, such as C₁₋₄ alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl—and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkyl sulfonyl groups, arylsulfonyl groups, —NH—NH₂; ═N—H; ═N-alkyl; —SH; —S-alkyl; —NH—C(O)—; —NH—C(═N)— and the like. Other suitable linkers would be known to one of ordinary skill in the art.

In some embodiments, the one or more diagnostic moieties can be attached via a biodegradable linker. In some embodiments, the biodegradable linker comprises an acid sensitive, such as a hydrazone moiety. In certain embodiments, the linker comprises a biodegradable moiety attached to a linker.

Various other linkers known to those skilled in the art or subsequently discovered may be used in place of (or in addition to) —O(CH₂)₃S(CH₂)₂NH₂. These include, for example, bifunctional linker groups such as alkylene diamines (H₂N—(CH₂)ᵣ—NH₂), where r is from 2 to 12; aminoalcohols (HO—(CH₂)ᵣ—NH₂), where r is from 2 to 12; aminothiols (HS—(CH₂)ᵣ—NH₂), where r is from 2 to 12; amino acids that are optionally carboxy-protected; ethylene and polyethylene glycols (H—(O—CH₂—CH₂)ₙ—OH, where n is 1-4). Suitable bifunctional diamines include ethylenediamine, 1,3-propanediamine, 1,4-butanediamine, spermidine, 2,4-diaminobutyric acid, lysine, 3,3′-diaminodipropylamine, diaminopropionic acid, N-(2-aminoethyl)-1,3-propanediamine, 2-(4-aminophenyl)ethylamine, and similar compounds. One or more amino acids also can be employed as the bifunctional linker molecule, such as β-alanine, γ-aminobutyric acid or cysteine, or an oligopeptide, such as di- or tri-alanine.

Other bifunctional linkers include:
—NH—(CH₂)ᵣ—NH—, where r is from 2-5,
—O—(CH₂)ᵣ—NH—, where r is from 2-5,
—NH—CH₂—C(O)—,
—O—CH₂—CH₂—O—CH₂—CH₂—O—,
—NH—NH—C(O)—CH₂—,
—NH—C(CH₃)₂C(O)—,
—S—(CH₂)ᵣ—C(O)—, where r is from 1-5,
—S—(CH₂)ᵣ—NH—, where r is from 2-5,
—S—(CH₂)ᵣ—O—, where r is from 1-5,
—S—(CH₂)—CH(NH₂)—C(O)—,
—S—(CH₂)—CH(COOH)—NH—,
—O—CH₂—CH(OH)—CH₂—S—CH(CO₂H)—NH—,
—O—CH₂—CH(OH)—CH₂—S—CH(NH₂)—C(O)—,
—O—CH₂—CH(OH)—CH₂—S—CH₂—CH₂—NH—,
—S—CH₂—C(O)—NH—CH₂—CH₂—NH—, and
—NH—O—C(O)—CH₂—CH₂—O—P(O₂H)—.

Examples of constructs useful in the present invention include mannosylamino dextrans (MAD) such as tilmanocept and m-tilmanocept. In some embodiments, the dextran-based moiety having at least one CD206 targeting moiety attached thereto can be a compound of Formula (I):

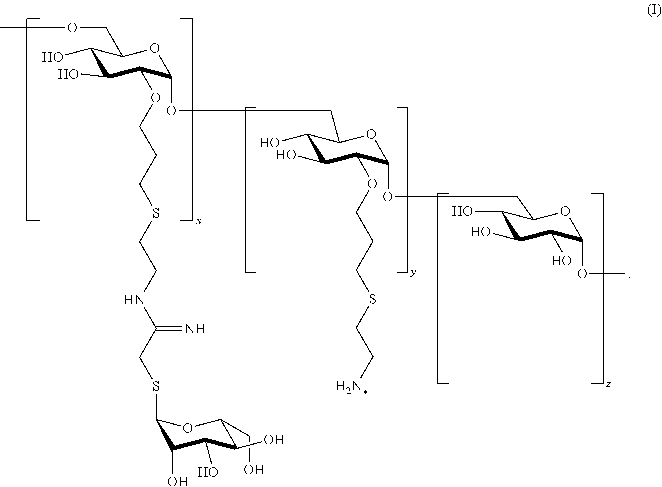

wherein the * indicates the point at which a diagnostic moiety can be attached. In certain embodiments, a diagnostic moiety can be attached via a linker. In certain embodiments, x can be between about 10 to about 25, about 5 to about 25, about 10 to about 20, about 15 to about 25, about 15 to about 20 and ranges therebetween. In some embodiments, y can be between about 35 and about 70, about 40 and about 70, about 50 and about 65, and ranges therebetween. In some embodiments, z can be between about 40 to about 70, about 50 to about 65, about 50 to about 60 and ranges therebetween.

In other embodiments, the compound of the present invention can be a compound of Formula (II):

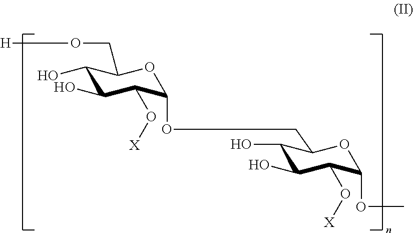

### Wherein

each X is independently H, L₁-A, or L₂-R; each L₁ and L₂ are independently linkers;
each A independently comprises a detection label or H;
each R independently comprises a CD206 targeting moiety or H; and
n is an integer greater than zero.

In certain embodiments, L₁ is a linker as described above. In certain embodiments, L₂ is a linker as described above.

In some embodiments a dosage of a compound described herein can comprise between about 5-500 μg of the compound, between about 200-300 μg of the compound, between about 100-300 μg of the compound, between about 100-200 μg of the compound, about 50-400 μg of the compound, about 125-175 μg of the compound, about 150 μg of the compound and ranges therebetween. In certain embodiments, the amount of radiolabeling can be altered to affect the radioactivity of a dose. For example, the radioactivity of about 0.1-50 mCi, about 0.5-10 mCi, about 10-50 mCi, about 10 mCi, about 5-25 mCi, about 1-15 mCi, and ranges therebetween.

### Synthesis

The compounds of this invention can be prepared by employing reactions as shown in the disclosed schemes, in addition to other standard manipulations that are known in the literature, exemplified in the experimental sections or clear to one skilled in the art. The following examples are provided so that the invention might be more fully understood, are illustrative only, and should not be construed as limiting. For clarity, examples having fewer substituents can be shown where multiple substituents are allowed under the definitions disclosed herein.

It is contemplated that each disclosed method can further comprise additional steps, manipulations, and/or components. It is also contemplated that any one or more step, manipulation, and/or component can be optionally omitted from the invention. It is understood that a disclosed method can be used to provide the disclosed compounds. It is also understood that the products of the disclosed methods can be employed in the disclosed compositions, kits, and uses.

The compounds of the present invention may be synthesized by any number of ways known to one of ordinary skill in the art. For example, linker 2 can be synthesized by opening succinic anhydride ring by tert-butyl carbazate. The resulting carboxylic acid is converted to the corresponding N-hydroxy succinimide (NHS) ester using EDC coupling reagent. MAD is then functionalized with linker 2 by forming an amide linkage. Then, the Boc protecting group can be removed under dilute acidic condition (typically 30-40% trifluoroacetic acid in DMSO) to obtain 4. Dilute acidic condition is required to avoid any unwanted cleavage of the glycosidic linkage present in dextran backbone. The resulting functionalized MAD can be purified by size exclusion filtration.

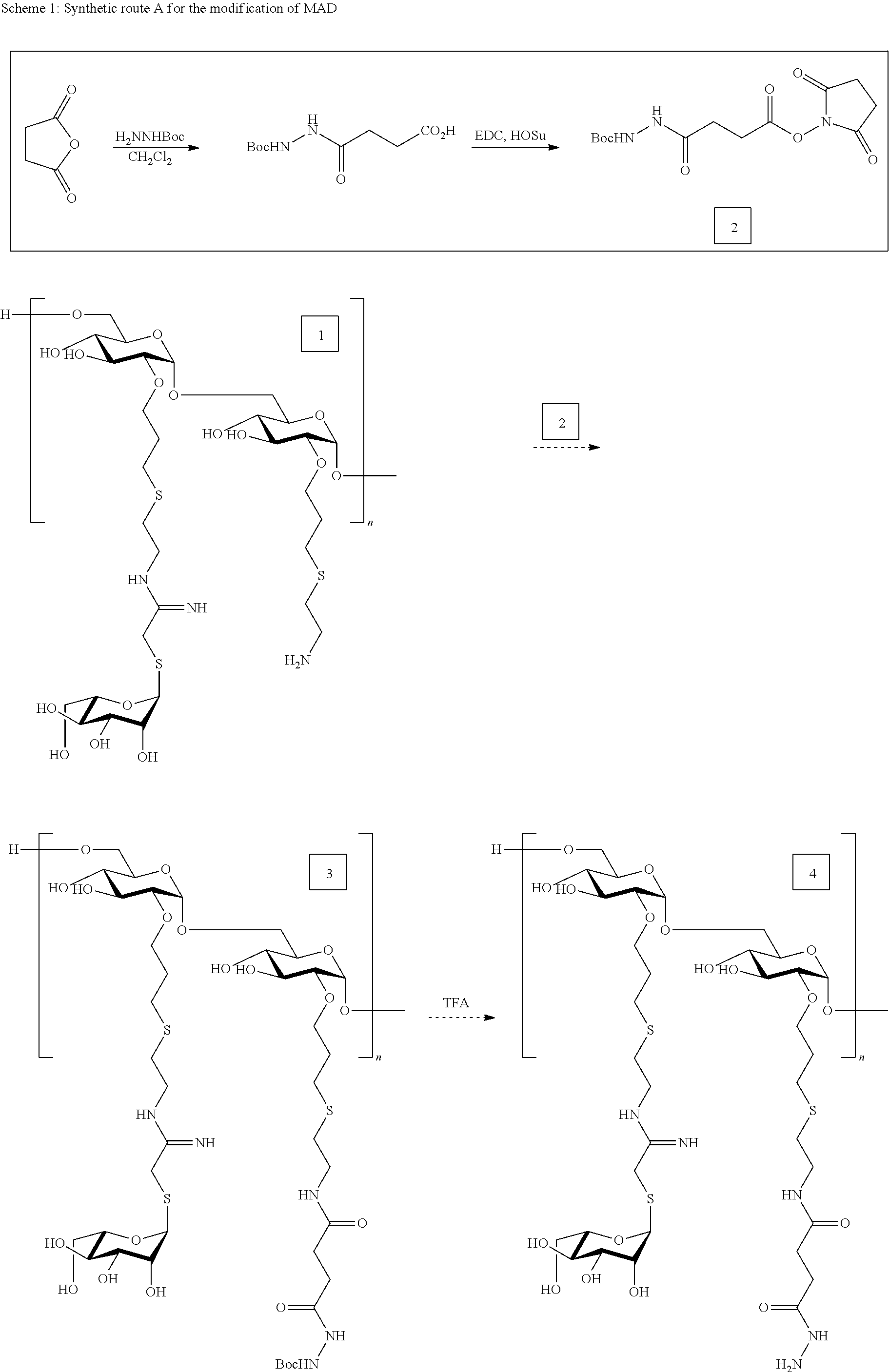

Alternatively, compounds according to the present invention may be synthesized according to Scheme 2. Free primary amine groups of MAD can be reacted with an excess of lactone under anhydrous condition. Unreacted lactone can be removed under reduced pressure to obtain modified MAD 6. The corresponding hydrazine derivative 7 can be prepared by reductive amination reaction using sodium cyanoborohydride or sodium triacetoxy borohydride as the reducing agent.

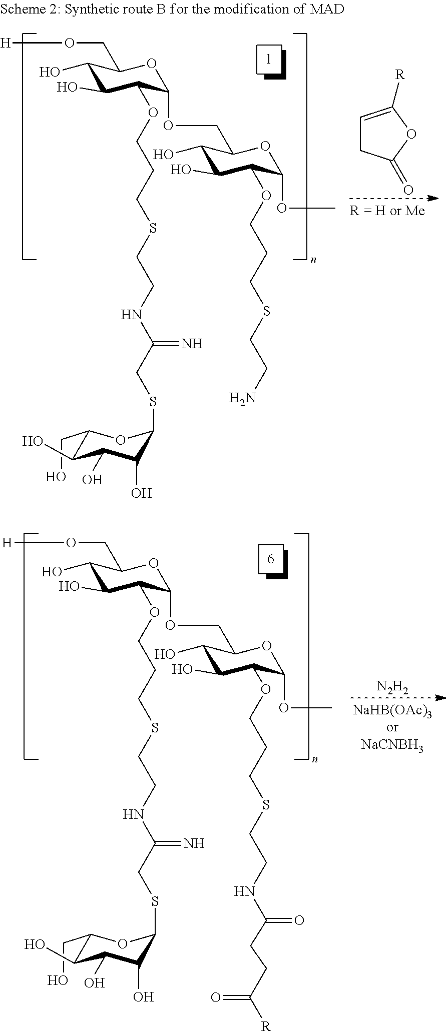

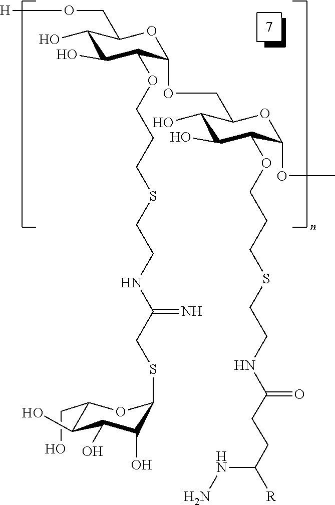

The conjugation of diagnostic moiety to MAD derivatives 4 or 7 can be as is shown in Scheme 3. MAD derivative 4 or 7 can be conjugated to a diagnostic moiety by formation of hydrazone linkage under anhydrous acidic condition or aqueous acidic conditions.

One of ordinary skill in the art may recognize other ways to synthesize the compounds of the present invention in view of the present disclosure.

As used herein, mannosylated dextran molecular constructs (MDMCs) can be a class of compounds sharing the following characteristics:
1. A backbone comprised of dextran,
2. Molecular leashes attached to the glucose moieties of the dextran backbone,
3. Mannose sugars attached to a portion of the molecular leashes,
4. One or more detection moieties attached to molecular leashes that are not occupied by mannose moieties,
5. Other sugar moieties in addition to mannose may optionally be added to molecular leashes that are not occupied by either mannose moieties or the detection moieties.

For certain MDMCs, the size of the backbone (e.g., dextran backbone), the number and molecular structure of the leashes, mannose moieties, detection moieties, and other added components (e.g., sugars) can be varied by design and these variations would be envisaged by the skilled artisan in view of the description contained herein. MDMCs can be constructs designed to deliver small molecule payloads of detection moieties to CD206 expressing cells. In certain embodiments, MDMCs can be designed to deliver detection moieties to CD206 expressing cells residing in atherosclerotic plaques. More specifically, MDMCs can be designed to deliver detection moieties to CD206 expressing macrophages residing in atherosclerotic plaques.

In certain embodiments, non-invasive macrophage-specific molecular imaging utilizing a MDMC can be used to image or visualize aortic and/or vascular inflammation. More specifically, the imaged or visualized aortic and/or vascular inflammation resides in atherosclerotic plaques. In some embodiments, MDMC based imaging enables quantification of aortic and/or vascular inflammation. In some embodiments, the structural mannose elements of MDMCs can direct radiolabeled MDMCs to the CD206+ macrophages where the activated types of macrophages express high levels of CD206. ^{99m}Tc-Tilmanocept is an example of a MDMC (FIG. 1). More specifically, such CD206-expressing macrophages internalize ^{99m}Tc-tilmanocept in a manner that is cumulative, not down-regulated by a process of pinocytosis that allows discriminating ^{99m}Tc signal to accumulate at sites of vascular inflammation such as non-calcified atherosclerotic plaques. Given the specificity of ^{99m}Tc-tilmanocept for binding CD206+ macrophages, aortic inflammation quantified in this manner may be considered to reflect in situ arterial CD206+ macrophage infiltration. Recognition that macrophages promote atherosclerotic plaque progression has provided impetus for the development of more specific non-invasive molecular techniques to functionally image arterial inflammation and needs exist to address such shortcomings. Such techniques might be expected to help identify patients at risk for clinical atherosclerosis mediated CVD cardiovascular events and to track the response of such patients to anti-inflammatory atherosclerosis targeted therapies.

An imaging study was conducted involving 9 subjects. All subjects had Framingham Risk Scores (FRS) indicating an intermediate risk of experiencing a CVD event (MI or IS). None of the subjects were symptomatic for CVD at the time of imaging study. All subjects were expected to have some level of atherosclerotic plaque burden due to their intermediate FRS. Six of the subjects were HIV+ and three were HIV−. This distribution of HIV+ and HIV− subjects was chosen because prior to imaging it was expected that the HIV+ subjects would have a higher atherosclerotic plaque burdens than the HIV− subjects, permitting the study to evaluate subjects with a broad range of plaque burdens. In each of the subjects, systemic administration of ^{99m}Tc-tilmanocept resulted in markedly increased ^{99m}Tc-tilmanocept uptake in the vascular system, in some embodiments the aorta. Among the HIV− subjects aortic uptake of ^{99m}Tc-tilmanocept was particularly striking (FIG. 2). Indeed, among the HIV− subjects', high-level ^{99m}Tc-tilmanocept (greater than 40-times the unaffected vascular tissue) uptake was detectable across and average of 20.4% of the aortic surface volume. Among HIV− subjects well-matched to a HIV+ cohort on FRS, high-level ^{99m}Tc-tilmanocept uptake can be detectable across an average of 4.3% of the aortic surface volume (FIG. 3). These results are consistent with the pre-imaging expectation that HIV− subjects would have less inflamed atherosclerotic plaque involvement than HIV+ subjects.

Context on the clinical significance of aortic ^{99m}Tc-tilmanocept uptake among HIV+ subjects can be demonstrated by findings from two contemporaneously performed non-invasive imaging studies: CTA and [18F]Na-PET/CT scanning. CTA achieves quantification of aortic and coronary subclinical plaque volume—including non-calcified plaque volume, grossly calcified plaque volume, and total plaque volume. [18F]Na-PET/CT scanning allows for more precise identification of areas of active arterial/plaque calcification. A significant relationship between aortic volume with high-level ^{99m}Tc-tilmanocept uptake the volume of non-calcified aortic plaque can be identified (r=0.078, p=0.01) (FIG. 3F). A pathophysiologic link between arterial inflammation and plaque volume in individuals who are asymptomatic for CVD can be identified using embodiments disclosed herein. Comparing aortic ^{99m}Tc-tilmanocept uptake and aortic [18F]Na uptake among individual index subjects, demonstrated spatially dysynchronous uptake (FIG. 4). Such dysynchrony demonstrates that ^{99m}Tc-tilmanocept SPECT scanning preferentially identifies areas in atherosclerotic plaques that are actively involved in inflammatory processes, which implies areas of plaque that are more likely rupture and cause CVD events than calcified plaque areas.

Strengths of the present study include the first-in-human demonstration of a macrophage-specific, noninvasive imaging strategy to quantify arterial macrophage infiltration, utilizing systemic administration of ^{99m}Tc-Tilmanocept followed by SPECT/CT scanning. This novel functional arterial imaging technique represents an advance over existing techniques, such as [18F]FDG-PET/CT scanning, which are not macrophage specific. The technique was well tolerated by all subjects, with no drug-related adverse events. Levels of radiation administered in conjunction with the technique were lower than administered with standard atherosclerosis imaging techniques such as CTA.

Context on the biologic significance of aortic ^{99m}Tc-tilmanocept uptake in humans comes from convincing ex vivo co-localization experiments performed on NNCT and NDRI human aortic samples. In these experiments, aortic samples were probed with anti-CD206 antibodies and Alexa-488 labeled tilmanocept. These experiments show a very high-level of co-localization between anti-CD206 and Alexa-488-tilmanocept on cells in aortic sections, with a similar degree of co-localization in samples from HIV+ and HIV− individuals (for example, 92 vs. 89%) (FIG. 5). These experiments also showed that aortic CD206+ macrophages are also most often positive for the activated macrophage marker CD163 (about 90% in samples from HIV+ individuals; about 88% in samples from HIV− individuals). Together, these studies show that tilmanocept binds to CD206+ cells with high specificity and that the very large majority of CD206+ cells to which tilmanocept binds in aortic samples are activated macrophages, supporting the contention that the extent of ^{99m}Tc-tilmanocept localization to atherosclerotic plaques is proportional to the number of activated macrophages residing in the plaque.

Compositions as discussed herein may be used for diagnostic uses for conditions associated or related to vascular plaque and/or arterial inflammation. Some embodiments may comprise uses of imaging arterial inflammation. Some embodiments may comprise

### EXAMPLES

### Overall Study Design

The primary aim of this study was to determine whether in vivo quantification of CD206+ aortic macrophage infiltration in humans could be accomplished non-invasively by thoracic single photon emission computed tomography (SPECT)/CT scanning following subcutaneous administration of ^{99m}Tc-tilmanocept. To contextualize the clinical significance of aortic ^{99m}Tc-tilmanocept uptake, two additional non-invasive cardiovascular imaging tests were performed—computed tomography angiography (CTA) and [18F]Na-PET/CT scanning. Moreover, facilitating the identification of clinical parameters relating to aortic ^{99m}Tc-tilmanocept uptake, subjects underwent detailed history. The results of this study have been published^{[82]}.

### Example 1

Six HIV+ subjects with known subclinical atherosclerosis based on prior CTA were recruited and enrolled. Three HIV− subjects matched on Framingham Risk Score (FRS) were also recruited and enrolled. None of the subjects in either group had known current or prior clinical cardiovascular disease.

### Subject Inclusion/Exclusion Criteria and Enrollment

For both groups of study, subjects HIV+ and HIV− who were 18 years of age or older. Exclusion criteria included history of myocardial infarction, stable or unstable angina, or coronary artery stenting or surgery; current treatment with prescription systemic steroids or anti-inflammatory/immune suppressant medical therapies; any recent treatment with statin therapy; estimated glomerular filtration rate <60 ml/min/1.73 m² calculated by CDK-EPI; known allergy to dextrans and/or DTPA and/or radiometals; known severe allergy to iodinated contrast media; clinical contraindication to beta-blockers or nitroglycerin (administered during CTA); significant radiation exposure received within the past 12 months; and BMI>35 kg/m2 (due to scanner limitations). For HIV+ subjects, entry criteria also included documented HIV infection, current use of antiretroviral therapy with no changes to regimen within the last 3 months, and subclinical atherosclerosis demonstrable on CTA. Three HIV− subjects were enrolled with selection based on above criteria. HIV− subjects were selected with similar Framingham Risk Score to those among the HIV-infected subjects to ensure comparable CV risk among the groups. Eligible subjects underwent the study procedures as described herein.

### Results:

### Baseline Cardiometabolic Parameters

The mean age of study subjects was 58±5 years (mean±SD) and BMI was 24.1±4.3 kg/m². Baseline total cholesterol was 178±18 mg/dL, HDL was 50 (44, 63) mg/dL [median(IQR)], LDL was 104±12 mg/dL. No subject received statin therapy within one year of the study. The Framingham Risk Score did not differ significantly between groups (11.0% vs. 10.3%, P=0.62; HIV+ vs. HIV−, Table 1). Among the HIV+ subjects, the duration since HIV diagnosis was 23.5±8.0 years, the log₁₀ VL was 1.4 (1.3, 2.8) copies/mL, and the CD4+ T cell count was 534±138 cells/mm³ (Table 1).

TABLE 1
Baseline demographics, immune/inflammatory markers and flow
cytometry in HIV- infected and non-HIV-infected subjects

| | HIV-infected | | Non-HIV-infected | |
|---|---|---|---|---|
| | subjects | | subjects | |
| Parameter | (N = 6) | | (N = 3) | |
| Baseline demographics | | | | |
| Race | | | | |
| Black (%) | 33.3 | (2/6) | 0 | (0/3) |
| White (%) | 66.7 | (4/6) | 100 | (3/3) |
| Age (years) | 57.5 ± 5.5 | | 58.3 ± 5.9 | |
| BMI (kg/m2) | 23.1 ± 5.1 | | 25.9 ± 1.7 | |
| Current Smoking (%) | 33.3 | (2/6) | 0 | (0/3) |
| History of Smoking (%) | 50 | (3/6) | 67 | (2/3) |
| Hypertension | 16.7 | (1/6) | 0 | (0/6) |
| Creatinine (mg/dL) | 0.79 | (0.73, 1.1) | 0.84 ± 0.2 | |
| 10-Year ASCVD Score (%) | 6.9 ± 2.6 | | 6.1 ± 2.4 | |
| Framingham Point Score | 6.9 ± 2.6 | | 10.7 ± 1.5 6.1 ± 2.4 | |
| Total Cholesterol (mg/dL) | 174 ± 15 | | 187 ± 23 | |
| HDL Cholesterol (mg/dL) | 174 ± 15 | | 62 ± 20 | |
| LDL Cholesterol (mg/dL) | 105 ± 12 | | 114 | (81, 114) |
| Family History/ | 50 | (3/6) | 0 | (0/3) |
| Premature CHD (%) | | | | |
| HIV specific parameters | | | | |
| Log VL (copies/ml) | 1.4 | (1.3, 2.8) | | |

| | |
|---|---|
| CD4 Count (cells/mm³) | 534 ± 138 |
| Duration Since | 23.5 ± 8.0 |
| HIV Diagnosis (yrs) | |

Areas of ^{99m}Tc-Tilmanocept Uptake on SPECT/CT

For all subjects, ^{99m}Tc-tilmanocept SPECT/CT images were acquired within an image acquisition window extending from the middle of the neck to the most inferior portion of the liver, including a portion of the kidneys. Among all subjects, ^{99m}Tc-tilmanocept uptake was visualized specifically in three areas: the kidneys, liver, and aorta (FIG. 6). Cells the normally express CD206 are known to reside in the liver and kidneys. The high ^{99m}Tc-tilmanocept uptake in these areas stands in contrast to the low uptake of ^{99m} Tc-tilmanocept observed in other areas, such as the muscle. The liver uptake was similar among HIV-infected and non-HIV-infected subjects (the ratio of ^{99m}Tc-tilmanocept uptake in the liver to muscle was 5.7 (5.5, 8.1) in the HIV-infected group vs. 5.1 (4.2, 5.8) in non-HIV-infected group (P=0.37)).

Aortic ^{99m}Tc-Tilmanocept Uptake on SPECT/CT

Among all study subjects, ^{99m}Tc-tilmanocept uptake by SPECT/CT was specifically observed in the aortic arch (FIG. 2). Using ^{99m}Tc-tilmanocept uptake in the muscle as a surrogate for low-level ^{99m}Tc-tilmanocept activity in the venous blood pool, ^{99m}Tc-tilmanocept uptake for each anatomic region of interest was normalized to mean ^{99m}Tc-tilmanocept uptake in the muscle. To enhance specificity and avoid labeling areas of lower-level aortic ^{99m}Tc-tilmanocept uptake as significant in either group and to increase the stringency of the analysis, high-level ^{99m}Tc-tilmanocept uptake was defined as uptake greater than or equal to five-times mean muscle ^{99m}Tc-tilmanocept uptake, or approximately equal to the mean ^{99m}Tc-tilmanocept uptake observed in the liver. The burden of high-level ^{99m}Tc-tilmanocept uptake in the aorta was then quantified as the aortic volume with high-level ^{99m}Tc-tilmanocept uptake and as the percent aortic volume with high-level ^{99m}Tc-tilmanocept uptake. Overall, aortic volume with high-level ^{99m}Tc-tilmanocept uptake was observed to be significantly higher among the HIV+ subjects compared to the HIV− subjects [31,910 (19,922, 65, 745) vs. 8,276 [6,574, 10, 890) mm³], P=0.03; FIG. 3A). The percent aortic volume with high-level ^{99m}Tc-tilmanocept uptake was also significantly increased among the HIV-infected subjects compared to the non-HIV-infected subjects (20.4±9.5 vs. 4.3±0.7% P=0.009 FIG. 3B).

Aortic ^{99m}Tc-Tilmanocept Uptake in Relation to Atherosclerotic Plaque Evaluated by CTA and [18F]Na PET/CT

Aortic plaque volume and location were determined by CTA and related to aortic ^{99m}Tc-tilmanocept uptake by SPECT/CT (FIGS. 7A-D). HIV+ subjects had an increased volume of total aortic plaque (10,623.2±6746.7 vs. 2,271.3±1,148.5 mm³, P=0.03) and an increased volume of non-calcified aortic plaque (6,541.0±4697.3 vs. 1,371.0±900.2 mm³, P=0.04) compared with age-matched non-HIV-infected subjects with similar Framingham Risk Scores (FIG. 7E). In contrast, total aortic calcium score was not different between the groups (FIG. 7G). Volumes of total, non-calcified, and calcified coronary plaque were [432.1 (131.6, 813.7), 151.4 (52.4, 388.4), and 296.7±257.6 mm³, respectively] in the HIV+ group and [22.0 (0.0, 565.8), 4.3 (0.0, 246.1), and 112.5±179.7 mm³, respectively] in the non-HIV-infected group. Among all the study subjects, there was significant correlation between percent aortic volume with high-level ^{99m}Tc-tilmanocept uptake and the total volume of aortic atherosclerotic plaque (R=0.73, P=0.03). This correlation between ^{99m}Tc-tilmanocept aortic uptake and total plaque atherosclerotic volume as determined by CTA was largely driven by high-level ^{99m}Tc-tilmanocept uptake to non-calcified plaque as defined as atherosclerotic plaque with x-ray opacity of HU<130, (R=0.78, P=0.01; FIG. 7F). In contrast, the compositions and methods as described herein produced the unexpected results that percent aortic volume with high-level ^{99m}Tc-tilmanocept uptake was not significantly correlated (R=0.56, P=0.12) with calcified atherosclerotic plaque (HU >130). Subjects were also evaluated by [18F]Na PET/CT to further explore the relationships between ^{99m}Tc-tilmanocept localization (uptake) and the spatial distribution of calcified and non-calcified atherosclerotic plaque (FIG. 4). Some areas of co-localization of [18F]Na and high-level ^{99m}Tc-tilmanocept uptake were observed; however, most areas of [18F]Na uptake and high-level ^{99m}Tc-tilmanocept uptake were mutually distinct. Together, the images from both the CTA and [18F]Na PET/CT studies compared to the images obtained by ^{99m}Tc-tilmanocept SPECT/CT provided the surprising and unexpected results that ^{99m}Tc-tilmanocept administered systemically localizes to and is taken up predominately to non-calcified atherosclerotic plaques, enabling qualitative SPECT/CT imaging of the anatomical locations and volumes of non-calcified atherosclerotic plaques.

### Example 2—Ex Vivo Experiments on Tissue-Banked Aortic Samples from Individuals with and without HIV

Sections of aortas from 10 HIV+ and 10 HIV− individuals obtained from the National NeuroAIDS Tissue Consortium (NNTC) and the National Disease Research Institute (NDRI) were studied. Perhaps due to the chronic inflammation experienced by HIV+ individuals, the mean number of CD206+ cells/mm² was significantly higher in the aortic sections from HIV+ individuals as compared with the aortic sections from HIV− individuals (30.1±7.9 vs. 14.2±7.0 macrophages/mm², P=0.0002, FIGS. 5A and 5B). In samples from both HIV+ and HIV− individuals, the large majority of CD206⁺ cells also were shown to express the activated macrophage marker, CD163, confirming that the large majority of CD206+ cells were macrophages. The percentage of CD206⁺CD163⁺ macrophages in the aortic samples from HIV− infected and non-HIV-infected individuals was 90.0±6.3% vs. 88.3±4.5%, respectively (FIGS. 5D and 5F). In another set of experiments, sections of the aortas from HIV+ and HIV− individuals were probed with antibodies against CD206⁺ and fluorescently-labeled tilmanocept. The percentage of cells for which anti-CD206 antibodies and fluorescent tilmanocept localized (CD206+tilmanocept+) was 89.1±6.3% vs. 86.3±6.8% for the sample derived from HIV+ and HIV− individuals respectively (FIG. 5C, 5E). The percentage of CD206⁺ tilmanocept” macrophages was 7.8±7.0% in HIV+ individuals and 10.4±6.2% in HIV− individuals. Aortic samples from both groups also demonstrated a very low percentage of cells which were positive for tilmanocept but which were negative for CD206 (3.1±1.8% in HIV+ and 3.3±2.1% in HIV−) (FIG. 5E). These experiments demonstrate that the localization of tilmanocept in aortic samples is due primarily to tilmanocept binding to CD206 that is being expressed on activated macrophages. These results are relevant to the interpretation of the imaging results presented in Example 1 in which it was shown that ^{99m}Tc-tilmanocept localizes predominantly to non-calcified portions of atherosclerotic plaques. Together, the findings described from Example 1 and Example 2 demonstrate the unexpected and highly clinically significant result that ^{99m}Tc-tilmanocept localization and imaging findings are due to ^{99m}Tc-tilmanocept binding and internalization to CD206 expressed on macrophages occurring most numerously in non-calcified plaques. Thus, ^{99m}Tc-tilmanocept SPECT/CT imaging is not only providing the anatomical location and volume of non-calcified plaques, it is also providing information about the inflammatory microenvironment and specifically the extent of activated macrophage involvement in the inflammatory pathobiology occurring within atherosclerotic plaques. Atherosclerosis is a chronic, macrophage mediated, maladaptive inflammatory syndrome that causes death and disability through the rupture of non-calcified plaques. The ability to directly measure and monitory activated macrophages involvement in inflammation within non-calcified plaques has great significance for facilitating the management and monitoring of therapy for patients at risk of experiencing an atherosclerosis related CVD event.

### Methods for Study Procedures

Subjects underwent ^{99m}Tc-tilmanocept SPECT/CT and [18F]Na-PET/CT on the same day, and additionally underwent CTA on a separate day.

^{99m}Tc-Tilmanocept SPECT/CT

^{99m}Tc-Tilmanocept SPECT/CT was performed using a Symbia T6 SPECT/CT (Siemens, Hoffman Estates, Ill.). The SPECT/CT protocol was as follows: Prior to imaging, IV access was obtained in an arm or hand vein. A total dose of <2 mCi (50 micrograms) (1.37-1.85 mCI) of the radiotracer ^{99m}Tc-tilmanocept was injected subcutaneously by a physician trained in nuclear medicine. The total injected dose, site of injection, and timing of injection for each subject are reported in FIG. 9.

The six index HIV-infected subjects were studied first, followed by the control subjects. For the first index subject, a single injection was administered in the subcutaneous tissue between the extensor hallucis longus and the first extensor digitorum longus tendons in the right foot. For the second index subject, two injections in an analogous subcutaneous tissue space in the left foot were administered. For the third through ninth subjects (four additional HIV− infected index subjects and three non-HIV-infected subjects), single injections in analogous spaces of each foot were administered. For the first subject, SPECT/CT images were acquired after 20 minutes of ^{99m}Tc-Tilmanocept injection (30-minute scan duration) and after 94 minutes of ^{99m}Tc-Tilmanocept injection (60-minute scan duration). For the second subject, SPECT/CT images were acquired after 40 minutes (30-minute scan duration) and after 155 minutes (60-minute scan duration). As ^{99m}Tc-tilmanocept SPECT uptake was noted to be more robust at the second time point of image acquisition, for the next (third) subject, image acquisition commenced 126 minutes after injection (90-minute scan duration). For the next 6 subjects, image acquisition commenced ˜180-209 minutes after injection (90-minute scan duration). For each subject, positioning on the scanner table was analogous. Using the persistence scope, subjects were positioned so that the area of interest was included in the field of view. With the camera heads in H mode, SPECT acquisitions were performed using 2×120 views over 360 degrees for each camera head, step and shoot mode, 45 seconds per view of the thorax. Thoracic images extended from the middle of the neck to the most inferior portion of the liver, including a portion of the kidneys, for all subjects. All projections were acquired in two energy windows, namely [90-120 keV] and [126-154 keV]. After SPECT acquisition was finished, a CT scan of the subject was acquired on the same scanner, with bed geometries recorded, allowing for automatic co-registration of SPECT/CT. SPECT images were reconstructed using iterative ordered subsets expectation maximization algorithm (OSEM), with 8 subsets and 4 iterations with corrections made for Compton scatter and CT-based attenuation. The resulting reconstructed image volume was used to quantify target to background ratios using CT-guided volumes of interest (VOIs) drawn on areas of tilmanocept uptake of interest normalized to a reference region of interest. All image analysis was performed using the software AMIDE⁴⁶. For each subject, CT-guided volumes of interest were drawn to cover a large volume of the right lobe of the liver (156.0±66.5 cc), and to bilaterally cover the triceps. The ratio of mean muscle activity to mean liver activity was relatively consistent among all subjects (0.18±0.04), with no significant difference between groups (P=0.16). Due to subcutaneous route of tilmanocept injection, SUV calculations of tilmanocept uptake would not be appropriate, thus tilmanocept uptake in regions of interest were presented relative to tilmanocept uptake in a reference region. Muscle was chosen as a reference region for analysis, due to low and consistent activity across subjects and groups. For each subject, tilmanocept activity (uptake) was normalized to mean muscle activity of 1. Total aortic uptake of tilmanocept was analyzed on images normalized to muscle activity as follows: For each subject, a CT-guided VOI was hand-drawn to cover the entirety of the aorta visible in the SPECT field of view. This VOI extended from the aortic root through the aortic arch, and terminated in the descending aorta at the end of the SPECT field of view (near the most inferior portion of the liver). The mean size of the aortic VOI was 201±63 cc's for all subjects. To assure specificity of aortic analyses, “high-level” tilmanocept uptake was defined as any voxel (as that term is used with regard to VOI analysis and as one of skill in the art would understand that term in the context of the invention) with activity (uptake) at or above 5 times muscle activity. This level of tilmanocept uptake was roughly equivalent to the mean activity observed in the liver (FIG. 6). For each subject, the total volume within the aortic VOI that was at or above the “high-level” activity threshold was calculated, as was the percent of the total volume at or above that level.

### Computed Tomography Angiography (CTA)

CTA of the coronary arteries and thoracic aorta was performed with a Somatom Definition Flash 128-slice dual-source CT scanner (Siemens Medical Solutions, Forchheim, Germany) according to the guidelines of the Society of Cardiac Computed Tomography⁴⁷. The CT protocol included a non-contrast CT for calcium scoring, a timing bolus, and contrast-enhanced CTA. Immediately prior to CT, 0.6 mg of sublingual nitroglycerin was given for vasodilation. Z-axis coverage extended from above the aortic arch to the level of the diaphragm during an inspiratory breath hold. The non-contrast calcium score CT acquisition was prospectively ECG-gated with a tube voltage of 120 kV and tube current of 80 mAs; image reconstruction was at a 3 mm thickness with a 1.5 mm overlap, a soft tissue filtered back projection kernel (B35f), and a limited field of view <25 cm to optimize pixel resolution of calcified plaque. After a 20 cc timing bolus, 60-80 cc of intravenous contrast (Iopamidol 370 g/cc, Bracco Diagnostics, Inc, Princeton, N.J. USA) at a rate of 4.5-5.4 cc/s based on subject size was injected through an antecubital intravenous catheter. Each contrast bolus was followed by a 40 cc normal saline flush. Prospectively ECG-triggered or retrospectively ECG-gated CTA scan modes were employed, based on the heart rate. The following CTA acquisition parameters were applied: tube voltage of 100 kV; ECG dependent tube current modulation with reference 380 mAs; adaptive pitch 0.2-0.4 based on heart rate; collimation 128×0.6 mm; rotation time 280 ms; and temporal resolution 75 ms. Images were reconstructed with 5% intervals between 60% and 75% of the R-R interval or with 20 ms intervals between 200 and 440 ms after the R wave, a slice thickness of 0.75 mm, an increment of 0.4 mm, a soft tissue iterative reconstruction kernel (I31f), and a limited field of view <25 cm to optimize pixel resolution of aortic and coronary artery plaque.

Semi-automated measurement of coronary artery and thoracic aortic plaque volume on the CTA was performed using a dedicated 3D workstation (Aquarius iNtuition version 4.11, Terarecon, Foster City, Calif. USA). The software generated curved multiplanar long and short axis images through the artery after centerline detection, then generated contours of the inner luminal and outer arterial wall. Manual adjustment of luminal and wall boundaries was performed as needed. Plaque was defined as any visible focal or circumferential wall thickening; plaque length was established visually with calipers at the proximal and distal plaque limits. Noncalcified plaque volume was defined as voxels with a CT attenuation <130 Hounsfield Units (HU); total plaque volume was defined as voxels of any attenuation. This volumetric plaque measurement technique has previously been shown to have excellent intraobserver, interobserver, and interscan reproducibility for coronary plaque volume⁴⁸⁻⁵³.

The presence and extent of coronary artery and thoracic aortic calcification was assessed on the non-contrast calcium score CT and calculated using the Agatston method using a threshold of >130 Hounsfield Units (HU) to define calcified plaque⁵⁴. Minimum intensity projection (MinIP) and 3d volume rendered images of aortic arch plaque were generated using 3d workstations (AQi, Terarecon, Foster City, Calif. USA and SyngoVia, Siemens Healthcare, Erlangen Germany) for presentation purposes.

[18F]Na-PET/CT

[18F]Na-PET/CT was performed using a Siemens Biograph PET/CT 64 slice system (Siemens Medical Solutions, Knoxville, Tenn.). The PET/CT protocol was as follows: Prior to imaging, IV access was obtained in an arm or hand vein. An IV injection of ˜4 mCI of the radiotracer ¹⁸F-NaF was injected. The catheter was flushed post-injection with approximately 40 ml of saline solution. [18F]Na-PET/CT scanning commenced ˜60 minutes thereafter. The injected dose, location of injection, and timing of injection relative to scan per subject has been published^{[14]}. With respect to the [18F]Na-PET/CT scanning, subjects were positioned on the scanner table with support devices under the back and/or legs. After a lag time of approximately 60 minutes, PET images (from the mid skull to the lower portion of the liver in 2 to 3 bed positions) were acquired using the PET scanner based on anatomical landmarks from the scout CT. An attenuation correction CT scan (non-enhanced 120 kV and 50 mA) was first performed, followed by PET imaging of the neck and thorax for 20 min/bed position. Coincidence event data was acquired and stored in list mode format and compressed in sinogram space. Randoms and Compton scatter was estimated and incorporated, along with attenuation correction, in the iterative OSEM reconstruction algorithm. Images were reconstructed using OSEM with 4 iterations and 16 subsets. Corrections were performed for scatter, randoms, and attenuation. The maximum standard uptake value SUV, defined as the decay corrected tissue concentration of the tracer divided by the injected dose per body weight, was computed and corrected for blood pool activity in the superior vena cava.

Anthropometric Measurements:

Subjects' height and weight was measured and BMI was calculated.

Assays:

Creatinine, glucose, CBC, lipid levels and HbA1c were measured through standard chemistry and hematology labs using standard technique. For HIV-infected subjects, HIV viral load was determined by ultrasensitive RT PCR (Cobas Ampliprep/Cobas Taqman HIV-1 test; lower limit of detection, 20 copies/mL. For non-HIV-infected subjects, HIV testing was performed by ELISA (Abbott).

Ex Vivo Experiments on Tissue-Banked Aortic Samples from Individuals with and without HIV

Aortic samples from individuals with HIV (n=10) and without HIV (n=10) were obtained from the National NeuroAIDS Tissue Consortium (NNTC) and the National Disease Research Institute (NDRI). These aortic sections, in original form, were formalin-fixed and paraffin embedded. Processing of these sections was as follows: Sections were deparaffinized and rehydrated in xylenes and graded ethanols followed by antigen retrieval (Vector) using high heat for 20 minutes. Sections were then incubated with peroxidase block (Dako) and washed in TBS-T, followed by a 30 minute protein block.

Single Label Immunohistochemistry with Antibodies Against CD206 and CD163

Sections were incubated with monoclonal antibodies recognizing CD206 (R&D Systems) or CD163+ (Serotec) overnight at 4° C. (CD206) or for 1 hour at room temperature (CD163). Sectioned were washed in TBS-T and incubated with an anti-mouse secondary antibody conjugated to horseradish peroxidase (Dako). The reaction product was visualized using 3, 3′-diaminobenzidine tetrahydrochloride (DAB, Dako). The average number of immune positive macrophages/mm² plus or minus the standard deviation (SD) was determined by counting the number of positive macrophages from twenty non-overlapping 200x fields of view (field area=0.148 mm²) per section, using a Zeiss Axio Imager M1 microscope with Plan-Apochromat×20/0.8 Korr objectives (Carl Zeiss Microimaging Inc., Thornwood, N.Y.).

Double Label Immunofluorescence with Antibodies Against CD206 and CD163 and Fluorescently Labeled Tilmanocept

Sections were permeabilized in 0.1% TritonX-100/PBS/fish skin gelatin (FSG) and washed with PBS/FSG. They were subsequently blocked in PBS/FSG with 10% normal goat serum (NGS), followed by 1 hour of overnight incubation with primary antibodies diluted in PBS/FSG/normal goat serum. After primary incubation, sections were washed in PBS/FSG before adding the fluorescent secondary antibody diluted in PBS/FSG normal goat serum. Finally, the sections were washed in PBS/FSG and incubated in copper sulfate in ammonium acetate for 45 minutes to quench autofluorescence. Tissue sections were stained with combinations of CD206, CD163, and tilmanocept and visualized using a Zeiss Axio Imager.Z2 with Apotome filter and ×200 objective. For sections double labeled with CD206 and CD163 antibodies, the percentage of CD206⁺CD163⁺, CD206⁺CD163⁻, CD206⁻CD163⁺, and cells was determined by counting the number of positive cells for each phenotype divided by the total number of cells in the field of view. The average percentage was calculated from 20 random, non-overlapping ×200 fields of view. For sections double labeled with CD206 and tilmanocept, the percentage of CD206⁺ tilmanocept⁺, CD206⁺ tilmanocept⁻, and CD206⁻tilmanocept⁺ cells was determined by counting the number of positive cells for each phenotype divided by the total number of cells in the field of view. The average percentage was calculated from 20 random, non-overlapping ×200 fields of view.

### Statistical Considerations

The primary endpoint was aortic ^{99m}Tc-tilmanocept uptake on SPECT/CT scanning. Secondary endpoints were plaque on CTA, ¹⁸F-NaF uptake on PET/CT scanning, as well as immune and phenotypic data. Our initial goal was to determine feasibility of ^{99m}Tc-tilmanocept imaging in the group of HIV-infected subjects. Secondary aims were to compare ^{99m}Tc-tilmanocept uptake in the aorta among subjects in the HIV-infected group vs. the non-HIV− infected group. Our initial estimation was that with a sample size of 6 HIV-infected index subjects and 6 non-HIV-infected control subjects for primary comparison, assuming a sigma (SD of the mean) of 1, we would be able to detect a true difference of the means between the index group and the control group of 1.8 SD at 80% power, p<0.05. Based on the clear differences seen between the 6 HIV-infected subjects and the 3 non-HIV-infected control subjects investigated, results were reported after 9 subjects were studied. Subject demographic parameters, where continuous, were assessed for normality and presented as mean±SD or median (IQR). Between-group comparisons for aortic volume and percent aortic volume with high-level ^{99m}Tc-tilmanocept uptake were compared by the Wilcoxon rank test. Other comparisons were made using t-test for continuous variables, Wilcoxon test for non-continuous variables. Between-group comparisons for categorical variables were made using the Chi squared test. Correlations between SPECT uptake and CT/metabolic/immune parameters were assessed using Pearson's or Spearman's as appropriate. Analyses were performed using JMP (version 11; SAS Institute, Cary, N.C.). For the ex vivo experiments, t-test was used to compare mean number of CD206+ and CD163+ macrophages in the aortic samples from individuals with and without HIV. Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

The following list of references are incorporated in their entirety, and particularly, their disclosure related to diagnosis and treatment of anomalies and disease-states related to atherosclerotic and vascular plaque and the like.
1. Taleb S. Inflammation in atherosclerosis. Archives of cardiovascular diseases 2016.
2. Pelisek J, Eckstein H H, Zernecke A. Pathophysiological mechanisms of carotid plaque vulnerability: impact on ischemic stroke. Archivum immunologiae et therapiae experimentalis 2012; 60(6):431-442.
3. Burke A P, Virmani R. Pathophysiology of acute myocardial infarction. The Medical clinics of North America 2007; 91(4):553-572; ix.
4. Heron M. Deaths: Leading Causes for 2015. National vital statistics reports: from the Centers for Disease Control and Prevention, National Center for Health Statistics, National Vital Statistics System 2017; 66(5):1-76.
5. Barquera S, Pedroza-Tobias A, Medina C, Hernandez-Barrera L, Bibbins-Domingo K, Lozano R, et al. Global Overview of the Epidemiology of Atherosclerotic Cardiovascular Disease. Archives of medical research 2015; 46(5):328-338.
6. Soloperto G, Casciaro S. Progress in atherosclerotic plaque imaging. World journal of radiology 2012; 4(8):353-371.
7. Aronow H D, Beckman J A. Parsing Atherosclerosis: The Unnatural History of Peripheral Artery Disease. Circulation 2016; 134(6):438-440.
8. Kalbaugh C A, Kucharska-Newton A, Wruck L, Lund J L, Selvin E, Matsushita K, et al. Peripheral Artery Disease Prevalence and Incidence Estimated From Both Outpatient and Inpatient Settings Among Medicare Fee-for-Service Beneficiaries in the Atherosclerosis Risk in Communities (ARIC) Study. Journal of the American Heart Association 2017; 6(5).
9. Vashishtha D, McClelland R L, Ix J H, Rifkin D E, Jenny N, Allison M. Relation Between Calcified Atherosclerosis in the Renal Arteries and Kidney Function (from the Multi-Ethnic Study of Atherosclerosis). The American journal of cardiology 2017; 120(8):1434-1439.
10. Schneider M F, Gange S J, Williams C M, Anastos K, Greenblatt R M, Kingsley L, et al. Patterns of the hazard of death after AIDS through the evolution of antiretroviral therapy: 1984-2004. AIDS (London, England) 2005; 19(17):2009-2018.
11. Massanella M, Richman D D. Measuring the latent reservoir in vivo. The Journal of clinical investigation 2016; 126(2):464-472.
12. Pinto DSM, da Silva M. Cardiovascular Complications of Human Immunodeficiency Virus Infection. Current cardiology reviews 2017.
13. Subramanian S, Tawakol A, Burdo T H, Abbara S, Wei J, Vijayakumar J, et al. Arterial inflammation in patients with HIV. Jama 2012; 308(4):379-386.
14. Nou E, Lo J, Grinspoon S K. Inflammation, immune activation, and cardiovascular disease in HIV. AIDS (London, England) 2016; 30(10):1495-1509.
15. Nou E, Lo J, Hadigan C, Grinspoon S K. Pathophysiology and management of cardiovascular disease in patients with HIV. The lancet Diabetes & endocrinology 2016; 4(7):598-610.
16. Triant V A, Lee H, Hadigan C, Grinspoon S K. Increased acute myocardial infarction rates and cardiovascular risk factors among patients with human immunodeficiency virus disease. The Journal of clinical endocrinology and metabolism 2007; 92(7):2506-2512.
17. Triant V A. HIV infection and coronary heart disease: an intersection of epidemics. The Journal of infectious diseases 2012; 205 Suppl 3:S355-361.
18. Chow F C, Regan S, Zanni M V, Looby S E, Bushnell C D, Meigs J B, et al. Elevated ischemic stroke risk among women living with HIV infection. AIDS (London, England) 2018; 32(1):59-67.
19. Sico J J, Chang C C, So-Armah K, Justice A C, Hylek E, Skanderson M, et al. HIV status and the risk of ischemic stroke among men. Neurology 2015; 84(19):1933-1940.
20. Calza L. HIV Infection and Myocardial Infarction. Current HIV research 2016; 14(6):456-465.
21. Lang S, Boccara F, Mary-Krause M, Cohen A. Epidemiology of coronary heart disease in HIV-infected versus uninfected individuals in developed countries. Archives of cardiovascular diseases 2015; 108(3):206-215.
22. Freiberg M S, Chang C C, Kuller L H, Skanderson M, Lowy E, Kraemer K L, et al. HIV infection and the risk of acute myocardial infarction. JAMA internal medicine 2013; 173(8):614-622.
23. Mosser D M, Edwards J P. Exploring the full spectrum of macrophage activation. Nature reviews Immunology 2008; 8(12):958-969.
24. Wynn T A, Chawla A, Pollard J W. Macrophage biology in development, homeostasis and disease. Nature 2013; 496(7446):445-455.
25. Martinez F O, Gordon S. The M1 and M2 paradigm of macrophage activation: time for reassessment. F1000 prime reports 2014; 6:13.
26. Heftdal L D, Stengaard-Pedersen K, Ornbjerg L M, Hetland M L, Horslev-Petersen K, Junker P, et al. Soluble CD206 plasma levels in rheumatoid arthritis reflect decrease in disease activity. Scandinavian journal of clinical and laboratory investigation 2017; 77(5):385-389.
27. Andersen M N, Andersen N F, Rodgaard-Hansen S, Hokland M, Abildgaard N, Moller H J. The novel biomarker of alternative macrophage activation, soluble mannose receptor (sMR/sCD206): Implications in multiple myeloma. Leukemia research 2015; 39(9):971-975.
28. Wileman T E, Lennartz M R, Stahl P D. Identification of the macrophage mannose receptor as a 175-kDa membrane protein. Proceedings of the National Academy of Sciences of the United States of America 1986; 83(8):2501-2505.
29. Ezekowitz R A, Sastry K, Bailly P, Warner A. Molecular characterization of the human macrophage mannose receptor: demonstration of multiple carbohydrate recognition-like domains and phagocytosis of yeasts in Cos-1 cells. The Journal of experimental medicine 1990; 172(6):1785-1794.
30. Su Y, Bakker T, Harris J, Tsang C, Brown G D, Wormald M R, et al. Glycosylation influences the lectin activities of the macrophage mannose receptor. The Journal of biological chemistry 2005; 280(38):32811-32820.
31. Taylor M E, Drickamer K. Structural requirements for high affinity binding of complex ligands by the macrophage mannose receptor. The Journal of biological chemistry 1993; 268(1):399-404.
32. East L, Isacke C M. The mannose receptor family. Biochimica et biophysica acta 2002; 1572(2-3):364-386.
33. Martinez-Pomares L. The mannose receptor. Journal of leukocyte biology 2012; 92(6):1177-1186.
34. Chinetti-Gbaguidi G, Colin S, Staels B. Macrophage subsets in atherosclerosis. Nature reviews Cardiology 2015; 12(1):10-17.
35. Chistiakov D A, Bobryshev Y V, Nikiforov N G, Elizova N V, Sobenin I A, Orekhov A N. Macrophage phenotypic plasticity in atherosclerosis: The associated features and the peculiarities of the expression of inflammatory genes. International journal of cardiology 2015; 184:436-445.
36. Colin S, Chinetti-Gbaguidi G, Staels B. Macrophage phenotypes in atherosclerosis. Immunological reviews 2014; 262(1):153-166.
37. Gui T, Shimokado A, Sun Y, Akasaka T, Muragaki Y. Diverse roles of macrophages in atherosclerosis: from inflammatory biology to biomarker discovery. Mediators of inflammation 2012; 2012:693083.
38. Cochain C, Zernecke A. Macrophages in vascular inflammation and atherosclerosis. Pflugers Archiv: European journal of physiology 2017; 469(3-4):485-499.
39. Bobryshev Y V, Nikiforov N G, Elizova N V, Orekhov A N. Macrophages and Their Contribution to the Development of Atherosclerosis. Results and problems in cell differentiation 2017; 62:273-298.
40. Moore K J, Sheedy F J, Fisher E A. Macrophages in atherosclerosis: a dynamic balance. Nature reviews Immunology 2013; 13(10):709-721.
41. Wolfs I M, Donners M M, de Winther M P. Differentiation factors and cytokines in the atherosclerotic plaque micro-environment as a trigger for macrophage polarisation. Thrombosis and haemostasis 2011; 106(5):763-771.
42. Libby P. Inflammation in atherosclerosis. Arteriosclerosis, thrombosis, and vascular biology 2012; 32(9):2045-2051.
43. Sakakura K, Nakano M, Otsuka F, Ladich E, Kolodgie F D, Virmani R. Pathophysiology of atherosclerosis plaque progression. Heart, lung & circulation 2013; 22(6):399-411.
44. Liang M, Puri A, Devlin G. The vulnerable plaque: the real villain in acute coronary syndromes. The open cardiovascular medicine journal 2011; 5:123-129.
45. Bentzon J F, Otsuka F, Virmani R, Falk E. Mechanisms of plaque formation and rupture. Circulation research 2014; 114(12):1852-1866.
46. Akyildiz A C, Speelman L, van Velzen B, Stevens RRF, van der Steen AFW, Huberts W, et al. Intima heterogeneity in stress assessment of atherosclerotic plaques. Interface Focus 2018; 8(1):20170008.
47. Chistiakov D A, Myasoedova V A, Melnichenko A A, Grechko A V, Orekhov A N. Calcifying Matrix Vesicles and Atherosclerosis. Biomed Res Int 2017; 2017:7463590.
48. Chung J H, Lee J M, Her A Y, Cho H, Doh J H, Nam C W, et al. Plaque Characteristics and Ruptured Plaque Location according to Lesion Geometry in Culprit Lesions of ST-Segment Elevation Myocardial Infarction. Korean Circ J 2017; 47(6):907-917.
49. Jung S, Song S W, Lee S, Kim S H, Ann S J, Cheon E J, et al. Metabolic phenotyping of human atherosclerotic plaques: Metabolic alterations and their biological relevance in plaque-containing aorta. Atherosclerosis 2017; 269:21-28.
50. Sturlaugsdottir R, Aspelund T, Bjornsdottir G, Sigurdsson S, Thorsson B, Eiriksdottir G, et al. Predictors of carotid plaque progression over a 4-year follow-up in the Reykjavik REFINE-study. Atherosclerosis 2017; 269:57-62.
51. Wierer M, Prestel M, Schiller H, Yan G, Schaab C, Azghandi S, et al. Compartment-resolved Proteomic Analysis of Mouse Aorta during Atherosclerotic Plaque Formation Reveals Osteoclast-specific Protein Expression. Mol Cell Proteomics 2017.
52. Cheng D, Li X, Zhang C, Tan H, Wang C, Pang L, et al. Detection of vulnerable atherosclerosis plaques with a dual-modal single-photon-emission computed tomography/magnetic resonance imaging probe targeting apoptotic macrophages. ACS Appl Mater Interfaces 2015; 7(4):2847-2855.
53. Melzer S, Ankri R, Fixler D, Tarnok A. Nanoparticle uptake by macrophages in vulnerable plaques for atherosclerosis diagnosis. J Biophotonics 2015; 8(11-12):871-883.
54. Shioi A, Ikari Y. Plaque Calcification During Atherosclerosis Progression and Regression. Journal of atherosclerosis and thrombosis 2017.
55. Pugliese G, lacobini C, Blasetti Fantauzzi C, Menini S. The dark and bright side of atherosclerotic calcification. Atherosclerosis 2015; 238(2):220-230.
56. Miller J D. Arterial calcification: Conscripted by collagen. Nat Mater 2016; 15(3):257-258.
57. de Gaetano M, Crean D, Barry M, Belton O. M1- and M2-Type Macrophage Responses Are Predictive of Adverse Outcomes in Human Atherosclerosis. Frontiers in immunology 2016; 7:275.
58. Noguchi T, Nakao K, Asaumi Y, Morita Y, Otsuka F, Kataoka Y, et al. Noninvasive Coronary Plaque Imaging. J Atheroscler Thromb 2017.
59. Libby P. Inflammation in atherosclerosis. Nature 2002; 420(6917):868-874.
60. Hammoud D A. Molecular Imaging of Inflammation: Current Status. J Nucl Med 2016; 57(8):1161-1165.
61. Kolossvary M, Szilveszter B, Merkely B, Maurovich-Horvat P. Plaque imaging with CT-a comprehensive review on coronary C T angiography based risk assessment. Cardiovasc Diagn Ther 2017; 7(5):489-506.
62. Goel S, Miller A, Agarwal C, Zakin E, Acholonu M, Gidwani U, et al. Imaging Modalities to Identity Inflammation in an Atherosclerotic Plaque. Radiology research and practice 2015; 2015:410967.
63. Zanni M V, Toribio M, Wilks M Q, Lu M T, Burdo T H, Walker J, et al. Application of a Novel CD206+ Macrophage-Specific Arterial Imaging Strategy in HIV-Infected Individuals. J Infect Dis 2017; 215(8):1264-1269.
64. Kim E J, Kim S, Seo H S, Lee Y J, Eo J S, Jeong J M, et al. Novel PET Imaging of Atherosclerosis with 68Ga-Labeled NOTA-Neomannosylated Human Serum Albumin J Nucl Med 2016; 57(11):1792-1797.
65. Chen W, Dilsizian V. Targeted PET/CT imaging of vulnerable atherosclerotic plaques: microcalcification with sodium fluoride and inflammation with fluorodeoxyglucose. Current cardiology reports 2013; 15(6):364.
66. Valenti V, B O H, Heo R, Cho I, Schulman-Marcus J, Gransar H, et al. A 15-Year Warranty Period for Asymptomatic Individuals Without Coronary Artery Calcium: A Prospective Follow-Up of 9,715 Individuals. JACC Cardiovascular imaging 2015; 8(8):900-909.
67. Gibson A O, Blaha M J, Arnan M K, Sacco R L, Szklo M, Herrington D M, et al. Coronary artery calcium and incident cerebrovascular events in an asymptomatic cohort. The MESA Study. JACC Cardiovascular imaging 2014; 7(11):1108-1115.
68. Erbel R, Mohlenkamp S, Moebus S, Schmermund A, Lehmann N, Stang A, et al. Coronary risk stratification, discrimination, and reclassification improvement based on quantification of subclinical coronary atherosclerosis: the Heinz Nixdorf Recall study. Journal of the American College of Cardiology 2010; 56(17):1397-1406.
69. Polonsky T S, McClelland R L, Jorgensen N W, Bild D E, Burke G L, Guerci A D, et al. Coronary artery calcium score and risk classification for coronary heart disease prediction. Jama 2010; 303(16):1610-1616.
70. Gueret P, Deux J F, Bonello L, Sarran A, Tron C, Christiaens L, et al. Diagnostic performance of computed tomography coronary angiography (from the Prospective National Multicenter Multivendor EVASCAN Study). The American journal of cardiology 2013; 111(4):471-478.
71. Groothuis J G, Beek A M, Meijerink M R, Brinckman S L, Heymans M W, van Kuijk C, et al. Positive predictive value of computed tomography coronary angiography in clinical practice. International journal of cardiology 2012; 156(3):315-319.
72. Tarkin J M, Dweck M R, Evans N R, Takx R A, Brown A J, Tawakol A, et al. Imaging Atherosclerosis. Circ Res 2016; 118(4):750-769.
73. Brinjikji W, Huston J, 3rd, Rabinstein A A, Kim G M, Lerman A, Lanzino G. Contemporary carotid imaging: from degree of stenosis to plaque vulnerability. Journal of neurosurgery 2016; 124(1):27-42.
74. Nair S B, Malik R, Khattar R S. Carotid intima-media thickness: ultrasound measurement, prognostic value and role in clinical practice. Postgraduate medical journal 2012; 88(1046):694-699.
75. Ray A, Huisman M V, Rabelink T J. Can and should carotid ultrasound be used in cardiovascular risk assessment?: the internist's perspective. European journal of internal medicine 2015; 26(2):112-117.
76. Cope F O, Abbruzzese B, Sanders J, Metz W, Sturms K, Ralph D, et al. The inextricable axis of targeted diagnostic imaging and therapy: An immunological natural history approach. Nuclear medicine and biology 2016; 43(3):215-225.
77. Azad A K, Rajaram M V, Metz W L, Cope F O, Blue M S, Vera D R, et al. gamma-Tilmanocept, a New Radiopharmaceutical Tracer for Cancer Sentinel Lymph Nodes, Binds to the Mannose Receptor (CD206). Journal of immunology (Baltimore, Md.: 1950) 2015.
78. Vera D R, Wallace A M, Hoh C K, Mattrey R F. A synthetic macromolecule for sentinel node detection: (99 m) Tc-DTPA-mannosyl-dextran. Journal of nuclear medicine: official publication, Society of Nuclear Medicine 2001; 42(6):951-959.
79. Cheng K T, Vera D R, Wallace A M, Hoh C K, Mendez J, Leung K. 99mTc-Diethylenetriaminepentaacetic acid-mannosyl-dextran. In: Molecular Imaging and Contrast Agent Database (MICAD). Bethesda (Md.): National Center for Biotechnology Information (US); 2004.
80. Hosseini A, Baker J L, Tokin C A, Qin Z, Hall D J, Stupak D G, et al. Fluorescent-tilmanocept for tumor margin analysis in the mouse model. The Journal of surgical research 2014; 190(2):528-534.
81. Vera D R, Hall D J, Hoh C K, Gallant P, McIntosh L M, Mattrey R F. Cy5.5-DTPA-galactosyl-dextran: a fluorescent probe for in vivo measurement of receptor biochemistry. Nuclear medicine and biology 2005; 32(7):687-693.
82. Zanni M V, Toribio M, Wilks M Q, Lu M T, Burdo T H, Walker J, et al. Application of a Novel CD206+ Macrophage-Specific Arterial Imaging Strategy in HIV. The Journal of infectious diseases 2017.
83. Loening A M, Gambhir S S. AMIDE: a free software tool for multimodality medical image analysis. Molecular imaging 2003; 2(3):131-137.
84. Abbara S, Arbab-Zadeh A, Callister T Q, Desai M Y, Mamuya W, Thomson L, et al. SCCT guidelines for performance of coronary computed tomographic angiography: a report of the Society of Cardiovascular Computed Tomography Guidelines Committee. Journal of cardiovascular computed tomography 2009; 3(3):190-204.
85. Ferencik M, Mayrhofer T, Puchner S B, Lu M T, Maurovich-Horvat P, Liu T, et al. Computed tomography-based high-risk coronary plaque score to predict acute coronary syndrome among patients with acute chest pain—Results from the ROMICAT II trial. Journal of cardiovascular computed tomography 2015; 9(6):538-545.
86. Oberoi S, Meinel F G, Schoepf U J, Nance J W, De Cecco C N, Gebregziabher M, et al. Reproducibility of noncalcified coronary artery plaque burden quantification from coronary CT angiography across different image analysis platforms. AJR American journal of roentgenology 2014; 202(1):W43-49.
87. Nakazato R, Shalev A, Doh J H, Koo B K, Gransar H, Gomez M J, et al. Aggregate plaque volume by coronary computed tomography angiography is superior and incremental to luminal narrowing for diagnosis of ischemic lesions of intermediate stenosis severity. Journal of the American College of Cardiology 2013; 62(5):460-467.
88. Boogers M J, Broersen A, van Velzen J E, de Graaf F R, El-Naggar H M, Kitslaar P H, et al. Automated quantification of coronary plaque with computed tomography: comparison with intravascular ultrasound using a dedicated registration algorithm for fusion-based quantification. European heart journal 2012; 33(8):1007-1016.
89. Versteylen M O, Kietselaer B L, Dagnelie P C, Joosen I A, Dedic A, Raaijmakers R H, et al. Additive value of semiautomated quantification of coronary artery disease using cardiac computed tomographic angiography to predict future acute coronary syndrome. Journal of the American College of Cardiology 2013; 61(22):2296-2305.
90. Schuhbaeck A, Dey D, Otaki Y, Slomka P, Kral B G, Achenbach S, et al. Interscan reproducibility of quantitative coronary plaque volume and composition from CT coronary angiography using an automated method. European radiology 2014; 24(9):2300-2308.
91. Agatston A S, Janowitz W R, Hildner F J, Zusmer N R, Viamonte M, Jr., Detrano R. Quantification of coronary artery calcium using ultrafast computed tomography. Journal of the American College of Cardiology 1990; 15(4):827-832.

1. A compound comprising a dextran backbone having one or more CD206 targeting moieties and one or more diagnostic moieties attached thereto.

2. A compound according to claim 1, wherein the compound is a compound of Formula (II): 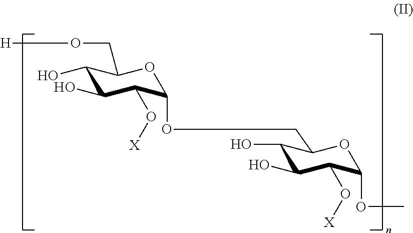
wherein
each X is independently H, L₁-A, or L₂-R; each L₁ and L₂ are independently linkers;
each A independently comprises a detection moiety or H; each R independently comprises a CD206 targeting moiety or H; and
n is an integer greater than zero; and
wherein at least one R is a CD206 targeting moiety and at least one A is a diagnostic moiety.

3. (canceled)

4. The compound of claim 1, wherein the at least one A is a gamma-emitting agent.

5. The compound of claim 1, wherein the at least one A is an isotope.

6. The compound of claim 1, wherein the at least one A is selected from the group consisting of ^{99m}Tc, ²¹⁰Bi, ²¹²Bi, ²¹³Bi, ²¹⁴Bi, ¹³¹Ba, ¹⁴⁰Ba, ¹¹C, ¹⁴C, ⁵¹Cr, ⁶⁷Ga, ⁶⁸Ga, ¹⁵³Gd, ⁸⁸Y, ⁹⁰Y, ⁹¹Y, ¹²³I, ¹²⁴I, ¹²⁵I, ¹³¹I, ¹¹¹In, ^{115m}In, ¹⁸F, ¹³N, ¹⁰⁵Rh, ¹⁵³Sm, ⁶⁷Cu, ⁶⁴Cu, ¹⁶⁶Ho, ¹⁷⁷Lu, ²²³Ra, ⁶²Rb, ¹⁸⁶Re and ¹⁸⁸Re, ³²P, ³³P, ⁴⁶Sc, ⁴⁷Sc, ⁷²Se, ⁷⁵Se, ³⁵S, ⁸⁹Sr, ¹⁸²Ta, ¹²³mTe, ¹²⁷Te, ¹²⁹Te, ¹³²Te, ⁶⁵Zn and ⁸⁹Zr, ⁹⁵Zr.

7.-12. (canceled)

13. A method of diagnosing inflammation within atherosclerosis comprising:
administering a compound to a subject comprising a dextran backbone having one or more CD206 targeting moieties and one or more diagnostic moieties attached thereto.

14. The method of claim 13, further comprising the step of quantifying the amount of non-calcified plaque in a subject's vascular tissues.

15. The method of claim 13, wherein said composition is non-invasive.

16. The method of claim 13, further comprising quantifying the subject's atherosclerotic non-calcified plaque amounts.

17. A composition for quantifying non-calcified atherosclerotic plaque, comprising:
^{99m}Tc-tilmanocept, wherein said composition is used for imaging and quantifying non-calcified atherosclerotic plaque.

18. A composition for non-invasive imaging of inflammation within atherosclerotic plaque, comprising:
^{99m}Tc-tilmanocept, wherein said composition is used for imaging inflammation within atherosclerotic plaque.

19. The composition of claim 18, wherein said subject is infected with human immunodeficiency virus (HIV).

20. A composition for non-invasively measuring inflammation within atherosclerotic plaque.

21. A composition comprising a diagnostic moiety for quantifying non-calcified plaque amounts in a subject.

22. The composition of claim 21, wherein said composition is non-invasive.

23. The composition of claim 21, further comprising quantifying the subject's non-calcified atherosclerotic plaque amounts.

24. A composition for measuring non-calcified plaque, comprising:
^{99m}Tc-tilmanocept, wherein said composition is used for imaging non-calcified plaque and measuring non-calcified plaque in a subject.

25. A composition for non-invasive imaging of atherosclerotic plaque, comprising:
99mTc-tilmanocept, wherein said composition is used for imaging atherosclerotic plaque.

26. The composition of claim 21, wherein said subject is infected with human immunodeficiency virus (HIV).

27. A composition for non-invasively measuring inflammation in atherosclerotic plaque.

28. A composition for quantifying non-calcified plaque in a subject.

29. The composition of claim 27, wherein the composition is non-invasive.

30. A mannosylated dextran molecular construct comprised of glucose moieties comprising:
a backbone comprised of dextran,
at least one leash attached to the glucose moieties of the dextran backbone,
at least one mannose sugars attached to a portion of the at least one leash; and
one or more diagnostic moieties attached to the at least one leash.

31.-32. (canceled)

33. A method of diagnosing inflammation in atherosclerotic plaque comprising administering the composition of claim 30.

34. A method of identifying non-calcified plaque in a subject comprising administering the composition of claim 30.

35. A method of quantifying non-calcified plaque in a subject comprising administering the composition of claim 30.

36. A method of quantifying the amount of non-calcified atherosclerotic plaque in a subject comprising administering the composition of claim 30.

37. The method of claim 30 further comprising the step of determining a subject's likelihood of developing cardiovascular disease.

38. A composition for imaging vascular inflammation, comprising:
a diagnostic moiety, wherein said composition is used for imaging vascular inflammation.

39. The compound of claim 1, wherein the at least one A is a contrast agent suitable for computed tomographic (CT) imaging.

40. The compound of claim 1, wherein the at least one A is selected from the group consisting of iodinated molecules, ytterbium and dysprosium.

41. The method of claim 13, further comprising imaging said subject using single-photon emission computed technology (SPECT/CT) wherein an image comprises visual indications of uptake of said compound in the subject's vascular tissues.

42. The method of claim 13, further comprising imaging said subject using planar gamma imaging wherein an image comprises visual indications of uptake of said compound in the subject's vascular tissues

43. A method of determining the anatomical location of non-calcified atherosclerotic plaque in a subject comprising:
administering a compound to a subject comprising a diagnostic moiety and one or more CD206 targeting moieties and one or more diagnostic moieties attached thereto.
